SEQUENCE LISTING (27 sequences)

SEQ ID NO: 1 - protein, 119 aa, Homo sapiens
EVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSS

SEQ ID NO: 2 - protein, 108 aa, Homo sapiens
DVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIKR

SEQ ID NO: 3 - DNA, 357 bp, Homo sapiens
  source 1..357: mol_type = other DNA
gaggtgcagctggtggagtctgggggaggcttggtaaagccgggggggtcccttagactctcctgtgcagcctctggcttcacttacagtaacgcctggatgagctgggtccgccaggctccagggaaggggctggagtgggttggccgtattaaaagcaaaactgatggtgggacaacagactacgctgcacccgtgcaaggcagattcaccatctcaagagatgattcaaaaaacacgctgtatctgcaaatgaacagcctgaaaaccgaggacacagccgtgtattactgtacgatttttggagtggttagctttgactactggggccagggaaccctggtcaccgtctcctca

SEQ ID NO: 4 - DNA, 324 bp, Homo sapiens
  source 1..324: mol_type = other DNA
gacgtccagatgacccagtctccatcctccctgtctgcatctgttggagacagagtcaccatcacttgccgggcaagtcagagcattagcgactatttaagttggtatcagcagagaccagggaaagcccctaacctcctgatctatgctgcatccaatttaaagactggggtcccatcaaggttcagtggcagtggatctgggacagatttcactctcaccatcagcactctgcaacctgaagattttgcaacgtactactgtcaacagagttacaggtccccgtggacgttcggccaagggaccaaggtggaaatcaaacga

SEQ ID NO: 5 - protein, 5 aa, Homo sapiens
NAWMS

SEQ ID NO: 6 - protein, 19 aa, Homo sapiens
RIKSKTDGGTTDYAAPVQG

SEQ ID NO: 7 - protein, 8 aa, Homo sapiens
FGVVSFDY

SEQ ID NO: 8 - protein, 11 aa, Homo sapiens
RASQSISDYLS

SEQ ID NO: 9 - protein, 7 aa, Homo sapiens
AASNLKT

SEQ ID NO: 10 - protein, 9 aa, Homo sapiens
QQSYRSPWT

SEQ ID NO: 11 - protein, 104 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTTTVT

SEQ ID NO: 12 - protein, 15 aa, Homo sapiens
YFDYWGQGTLVTVSS

SEQ ID NO: 13 - protein, 93 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYS

SEQ ID NO: 14 - protein, 13 aa, Homo sapiens
WTFGQGTKVEIKR

SEQ ID NO: 15 - protein, 1722 aa, Homo sapiens
MRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKEKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPDRAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYSGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNKSLMWFDKTPLSYTHWRAGRPTIKNEKFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKSKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHD

SEQ ID NO: 16 - protein, 30 aa, Homo sapiens
EVQLVESGGGLVKPGGSLRLSCAASGFTYS

SEQ ID NO: 17 - protein, 14 aa, Homo sapiens
WVRQAPGKGLEWVG

SEQ ID NO: 18 - protein, 32 aa, Homo sapiens
RFTISRDDSKNTLYLQMNSLKTEDTAVYYCTI

SEQ ID NO: 19 - protein, 11 aa, Homo sapiens
WGQGTLVTVSS

SEQ ID NO: 20 - protein, 23 aa, Homo sapiens
DVQMTQSPSSLSASVGDRVTITC

SEQ ID NO: 21 - protein, 15 aa, Homo sapiens
WYQQRPGKAPNLLIY

SEQ ID NO: 22 - protein, 32 aa, Homo sapiens
GVPSRFSGSGSGTDFTLTISTLQPEDFATYYC

SEQ ID NO: 23 - protein, 11 aa, Homo sapiens
FGQGTKVEIKR

SEQ ID NO: 24 - protein, 468 aa, Homo sapiens
MEWSWVFLFFLSVTTGVHSEVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 234 aa, Homo sapiens
MSVPTQVLGLLLLWLTDARCDVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 1404 bp, Homo sapiens
  source 1..1404: mol_type = other DNA
atggaatggagctgggtgttcctgttctttctgtccgtgaccacaggcgtgcattctgaagttcagctggtcgaaagcggaggaggtctggtgaaacccggtggctccctgaggctgagctgcgccgcctccggctttacttacagtaatgcctggatgtcctgggtcagacaggccccaggtaagggtctggagtgggtgggtaggattaagtctaaaactgatggcgggacaacagactatgccgccccagtgcaaggacggttcaccatttctagggacgactctaagaatacactgtatctgcagatgaacagcctcaaaacagaagacactgccgtttactactgtaccatctttggcgttgtctcctttgattattggggacagggtacactcgtgaccgtttcttccgcaagtacaaaggggccatcagtgtttccactggccccatcctctaagagcactagtggcggcacagccgccctgggatgtctggtgaaggactatttcccagagcctgtgaccgtcagctggaacagtggtgctctcacctcaggtgtgcacacattccccgctgtgctccaatccagtggcctctacagtctgagcagcgttgtgactgttcccagtagctcactgggcacccaaacctacatatgcaatgtgaaccataaacctagcaataccaaagtggacaagaaagtggaacctaagtcctgtgacaagactcatacctgtcctccttgtcctgccccagagctgctcggaggcccttccgtctttctcttcccaccaaagccaaaggataccctgatgatcagccggacacctgaggttacctgcgttgtggtcgacgtttcacacgaggatcctgaagtcaaattcaactggtacgttgatggagtcgaggtccacaacgccaaaaccaagcctcgcgaagaacaatacaatagcacatatagggtggtgtctgtgctcactgtcctgcaccaggactggctgaacggcaaggagtacaaatgcaaggttagtaacaaggccctgcccgcacccattgagaagactatcagtaaagctaagggccagcctcgcgagcctcaggtttacaccctgcctccctctagagaggaaatgacaaagaaccaggtgtctctcacctgcctggttaaaggattctatccatccgacattgctgtggaatgggaatccaacggacagcccgaaaacaactataagacaacaccacctgttctggattccgatggttccttctttctgtattccaaactcacagtggacaagagtcgctggcagcaaggtaacgtgttttcttgctccgtgatgcacgaagcactccacaatcactacactcagaagagtctcagcctctctccaggcaaa

SEQ ID NO: 27 - DNA, 702 bp, Homo sapiens
  source 1..702: mol_type = other DNA
atgtctgtgcctacccaggtgctgggactgctgctgctgtggctgacagacgcccgctgtgatgttcagatgacacagtctccaagtagtctcagcgcaagcgttggcgacagagtgactatcacatgcagagcctctcagtctatctctgactatctgtcttggtaccagcagaggccaggcaaagctccaaacctcctgatctatgctgccagtaatctgaagacaggcgtgcctagtagattctccgggtccggtagtgggactgatttcaccctgacaatctccacactgcaacctgaggattttgctacctactattgtcagcaatcttatcgcagcccttggaccttcggacaggggactaaggttgagattaaacgcaccgtggcagcacccagcgtctttatctttcctccctccgacgagcagctcaagtccggaacagcatcagtcgtttgcctcctgaataacttttatccaagggaggccaaggtccagtggaaagtcgacaatgccctccaatctggtaactcccaggagtctgtgactgaacaagattctaaggacagtacctattcactcagctccaccctgaccctcagcaaagcagactacgaaaagcataaagtttacgcttgcgaagtgacccaccaaggcctgtcttctcctgtcacaaagagttttaatagaggggagtgt

(19) United States
(12) Patent Application Publication
(10) Pub. No.: US 2025/0073346 A1
(43) Pub. Date: Mar. 6, 2025

(54) PHARMACEUTICAL COMBINATIONS

(71) Applicant: Oxford Bio Therapeutics Ltd, Oxford (GB)

(72) Inventors: Arnima BISHT, San Jose, CA (US); Christian ROHLFF, Oxford (GB); Abderranhim FANDI, Oxford (GB)

(21) Appl. No.: 18/711,146

(22) PCT Filed: Nov. 17, 2022
(86) PCT No.: PCT/GB2022/052913

§ 371 Date: May 17, 2024

Related U.S. Application Data
(60) Provisional application No. 63/280,916, filed on Nov. 18, 2021.

(51) Int. Cl. A61K 47/68; A61K 31/282; A61K 33/243; A61K 39/395; A61P 35/00

(52) U.S. Cl. CPC A61K 47/68033; A61K 31/282; A61K 33/243; A61K 39/3955; A61K 47/6849; A61P 35/00

(57) ABSTRACT

The present disclosure relates generally to the fields of immunology and molecular biology. More specifically, provided herein are pharmaceutical combinations comprising antibodies, or antigen-binding portions thereof, directed against LY75, and a platin; methods for preparing pharmaceutical combinations; and methods for the treatment of diseases, such as cancers mediated by LY75 expression or activity.

### INTRODUCTION

The present disclosure relates generally to the fields of immunology and molecular biology. More specifically, provided herein are pharmaceutical combinations comprising (A) antibodies, or antigen-binding portions thereof, directed against LY75, and (B) one or more platinum based antineoplastic drugs (platins); methods for preparing pharmaceutical combinations; and methods for the treatment of diseases, such as cancers mediated by LY75 expression or activity.

### BACKGROUND

Combination chemotherapy involves treating a patient with two or more different drugs simultaneously. The drugs may differ in their mechanism and side-effects. The biggest advantage of this is to minimize the chances of resistance development to any one agent. Furthermore, the drugs can often be used at lower doses, reducing toxicity.

There remains, however, a need for new treatments for cancers, and particularly for efficacious combination therapies.

Lymphocyte antigen 75 acts as an endocytic receptor to direct captured antigens from the extracellular space to a specialized antigen-processing compartment and is thought to cause a reduction in proliferation of B-lymphocytes. Expression of Lymphocyte antigen 75 has been observed in gastric, bladder, renal, pancreatic, ovarian, breast, colorectal, esophageal, skin, thyroid and lung cancers as well as Multiple Myeloma and many different subtypes of lymphomas and leukaemias. WO2009/061996 discloses isolated monoclonal antibodies which bind to human DEC-205 (LY75) and related antibody based compositions and molecules. Also disclosed are pharmaceutical compositions comprising the antibodies, as well as therapeutic and diagnostic methods for using the antibodies. WO2008/104806 discloses affinity reagents capable of binding to LY75 for use in the treatment or prophylaxis of cancer. WO2015/052537 discloses specific isolated antibodies capable of binding to LY75 and their use in the treatment various cancers.

Platinum compounds are one of the most widely used classes of drugs in cancer therapy and have remained the backbone of systemic cancer therapies since 1980. Approximately half of all patients on anticancer chemotherapy regimens are treated with a platinum drug. Cisplatin (cis-diamminedichloroplatinum(II)), the most potent platinum compound, was the first generation of platinum drugs approved by the FDA for the treatment of a wide spectrum of solid tumors. Cisplatin is taken into cells and reacts with intracellular macromolecules to form protein, RNA, and DNA adducts. Treatment with cisplatin often leads to an initial therapeutic response associated with complete disease remission, partial response or disease stabilization. However, cisplatin therapy requires additional medication to alleviate the side effects that are frequently associated with use, which include severe kidney problems, reduced immunity to infections, allergic reactions, gastrointestinal disorders, and hearing loss especially in younger patients. Despite these side effects and because of impressive potency, cisplatin is still used for the treatment of numerous human malignancies including bladder, head and neck, melanoma, lymphomas, and myelomas, non-small-cell lung cancer (NSCLC); small-cell lung cancer, and ovarian and testicular cancers. The major hurdle to the success of cisplatin is the drug resistance. Many tumors are intrinsically resistant to the platinum drug. Moreover, many sensitive tumors develop resistance gradually after initial response.

In order to reduce the dose-limiting toxicity of cisplatin, a second-generation of compounds was developed, for example, carboplatin (cis-diammine (1,1-cyclobutanecarboxylate) platinum (II)). The mechanism of action is similar to that of cisplatin, but carboplatin exhibits lower reactivity and slower DNA binding kinetics than cisplatin. Depending on the type of cancer, carboplatin may be between 8- and 45-fold less potent than cisplatin. With a reduced toxicity profile, carboplatin is suitable for more aggressive high-dose chemotherapy.

As resistance still remains a problem with the first- and second-generation compounds, the third-generation platinum drug oxaliplatin was developed to overcome such resistance. Oxaliplatin is a platinum complex with a (1R,2R)-1,2 diaminocyclohexane (DACH) ligand. Oxaliplatin is less reactive and has lower toxic side effects. The DACH ligand is more lipophilic, which increases the passive uptake of oxaliplatin above that seen with cisplatin and carboplatin. In combination with 5-FU and folinic acid, oxaliplatin currently has widespread approval for the treatment of adjuvant and metastatic colorectal cancer that is intrinsically insensitive to cisplatin. To date, only three platinum-based drugs that have entered clinical trials have gained international marketing approval, while another three (nedaplatin, lobaplatin, and heptaplatin) have gained approval in certain markets.

It has now been found that combinations of certain anti-LY75 antibodies with platins demonstrate synergistic results in the treatment of cancers.

### BRIEF SUMMARY OF THE INVENTION

In one aspect, the invention provides a pharmaceutical combination comprising:
an anti-LY75 antibody, or an antigen-binding portion thereof component, and a platin drug component.

In one embodiment, the pharmaceutical combination is in the form of a combined preparation for simultaneous, separate or sequential use.

In one embodiment the platin drug is cisplatin.

In one embodiment the platin drug is carboplatin.

In one embodiment the platin drug is oxaliplatin.

In one embodiment the platin drug is nedaplatin.

In one embodiment the platin drug is lobaplatin.

In one embodiment the platin drug is heptaplatin.

In one embodiment, the anti-LY75 antibody or antigen-binding portion thereof comprises a heavy chain variable region comprising 1, 2 or 3 CDRs selected from the group consisting of CDRs comprising SEQ ID NOs: 5, 6, and 7, and/or a light chain variable region comprising 1, 2 or 3 CDRs selected from the group consisting of CDRs comprising SEQ ID NOs: 8, 9 and 10.

In one embodiment, the anti-LY75 antibody, or an antigen-binding portion thereof, comprises:
a) a heavy chain variable region comprising:
   i) a first vhCDR comprising SEQ ID NO: 5;
   ii) a second vhCDR comprising SEQ ID NO: 6; and
   iii) a third vhCDR comprising SEQ ID NO: 7; and
b) a light chain variable region comprising:
   i) a first vICDR comprising SEQ ID NO: 8;
   ii) a second vICDR comprising SEQ ID NO: 9; and
   iii) a third vICDR comprising SEQ ID NO: 10;
optionally wherein any one or more of the above SEQ ID NOs independently comprise one, two, three, four or five amino acid substitutions, additions or deletions.

In one embodiment, any one or more of SEQ ID NOs: 5-10 independently comprise one, two, three, four or five conservative amino acid substitutions.

In a further embodiment, any one or more of SEQ ID NOs: 5-10 independently comprise one or two conservative amino acid substitutions.

In some embodiments, the anti-LY75 antibodies bind to LY75 (SEQ ID NO: 15) and are capable of being internalized by a cell expressing LY75.

In another embodiment, the anti-LY75 antibody comprises the heavy and/or light chain complementarity determining regions (CDRs) or variable regions (VRs) of the particular antibody described herein (e.g., referred to herein as “LY75_A1”). Accordingly, in one embodiment, the anti-LY75 antibody comprises the CDR1, CDR2, and CDR3 domains of the heavy chain variable (VH) region of antibody LY75_A1 having the sequence shown in SEQ ID NO: 1, and/or the CDR1, CDR2 and CDR3 domains of the light chain variable (VL) region of LY75_A1 having the sequence shown in SEQ ID NO:2.

In another embodiment, the anti-LY75 antibodies bind to human LY75 and include a heavy chain variable region comprising SEQ ID NO: 1, and/or conservative sequence modifications thereof.

The antibody may further include a light chain variable region comprising SEQ ID NO:2, and/or conservative sequence modifications thereof.

In a further embodiment, the anti-LY75 antibodies bind to human LY75 and include a heavy chain variable region and a light chain variable region including the amino acid sequences set forth in SEQ ID NOs: 1 and 2, respectively.

In one embodiment, the anti-LY75 antibody comprises a heavy chain variable region comprising SEQ ID NO:1 or a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 1.

In another embodiment, the anti-LY75 antibody comprises a light chain variable region comprising SEQ ID NO:2 or a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 2.

In another embodiment, the anti-LY75 antibody comprises a heavy chain framework region comprising an amino acid sequence that is at least 80%, at least 85%, at least 90%, at least 91%, at least 92%, at least 93%, at least 94%, at least 95%, at least 96%, at least 97%, at least 98%, at least 99% identical to the framework of the heavy chain variable region of SEQ ID NO: 1 as shown in SEQ ID NOS: 16, 17, 18 and 19.

In another embodiment, the anti-LY75 antibody comprises a light chain framework region comprising an amino acid sequence that is at least 80%, at least 85%, at least 90%, at least 91%, at least 92%, at least 93%, at least 94%, at least 95%, at least 96%, at least 97%, at least 98%, at least 99% identical to the framework of the light chain variable region of SEQ ID NO:2 as shown in SEQ ID NOS: 20, 21, 22 and 23.

In a further embodiment, the anti-LY75 antibody comprises a heavy chain comprising SEQ ID NO: 24 or a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 24. In another embodiment, the anti-LY75 antibody comprises a light chain comprising SEQ ID NO: 25 or a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 25. The heavy chain may comprise the sequences of SEQ ID NOs: 5-7 or 1. The light chain may comprise the sequences of SEQ ID NOs: 8-10 or 2.

In one embodiment, the anti-LY75 antibody competes for binding to LY75 with an antibody comprising heavy and light chain variable regions comprising the amino acid sequences set forth in SEQ ID NOs: 1 and 2, respectively (LY75_A1).

Other antibodies of the invention bind to the same epitope or an epitope on LY75 recognized by the antibodies described herein. In another particular embodiment, the antibody binds to an epitope on LY75 recognized by an antibody comprising heavy and/or light chain variable regions comprising the amino acid sequences set forth in SEQ ID NOs: 1 and 2, respectively, or amino acid sequences at least 80% identical thereto. In another embodiment, the antibody binds to an epitope on LY75 recognized by an antibody comprising heavy and/or light chain variable regions comprising the amino acid sequences set forth in SEQ ID NOs: 1 and 2 (LY75_A1).

In a further embodiment, the anti-LY75 antibodies comprise variable CDRs as compared to the parent antibodies described herein. Thus, the variant antibodies comprising variant variable regions of a parent antibody, wherein the parent antibody comprises a first vhCDR comprising SEQ ID NO:5, a second vhCDR comprising SEQ ID NO: 6, a third vhCDR comprising SEQ ID NO: 7, a first vICDR comprising SEQ ID NO:8, a second vICDR comprising SEQ ID NO:9 and a third vICDR comprising a SEQ ID NO: 10, and wherein the variant antibody has 1, 2, 3, 4, 5 or 6 amino acid substitutions collectively in the set of the first vhCDR, the second vhCDR, the third vhCDR, the first vICDR, the second vICDR and the third vICDR, with from 1 to 4, 1 to 3 or 1 to 2 substitutions of particular use, and wherein the antibody retains specific binding to LY75.

All of the antibodies disclosed herein can be full-length, for example, any of the following isotypes: IgG1, IgG2, IgG3, IgG4, IgM, IgA1, IgA2, IgAsec, IgD, and IgE. Alternatively, the antibodies can be fragments such as an antigen-binding portion or a single chain antibody (e.g., a Fab, F(ab′)₂, Fv, a single chain Fv fragment, an isolated complementarity determining region (CDR) or a combination of two or more isolated CDRs). The antibodies can be any kind of antibody, including, but not limited to, human, humanized, and chimeric antibodies.

In other embodiments, the anti-LY75 antibodies are in the form of an immunoconjugate (i.e., they further include a covalently-attached moiety). Preferably the covalently-attached moiety is a cytotoxic moiety.

In a particular embodiment, the covalently-attached moiety is a drug, such as a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an indolino-benzodiazepine, an amatoxin, CC1065 or taxol and derivatives thereof. In a preferred embodiment, the drug moiety is DM1 or DM4.

In one embodiment, the anti-LY75 antibody comprises a heavy chain variable region and a light chain variable region encoded by nucleic acid sequences comprising SEQ ID NOs: 3 and 4, respectively, or nucleic acid sequences having at least 85%, 86%, 87%, 88%, 89%, 90%, 91%, 92%, 93%, 94%, 95%, 96%, 97%, 98% or 99% identity to the aforementioned nucleic acid sequences or sequences which differ from SEQ ID NOs: 3 and 4 due to degeneracy of the genetic code.

In a further aspect there is provided a method of treating cancer in a patient comprising simultaneously, sequentially or separately administering to a patient in need thereof therapeutically-effective amounts of the components of the pharmaceutical combination of the invention.

In a further aspect of the present invention, there is provided a pharmaceutical combination of the invention for use in the treatment of cancer.

Also provided is the use of an anti-LY75 antibody, or an antigen-binding portion thereof as described herein, and a platin drug or a pharmaceutically-acceptable salt thereof in the manufacture of a pharmaceutical combination for simultaneous, separate or sequential use for the treatment of cancer.

In some embodiments, the cancer is selected from the list comprising pancreatic cancer, ovarian cancer, breast cancer, endometrial cancer, colorectal cancer, esophageal cancer, skin cancer, thyroid cancer, lung cancer (NSCLC and/or SCLC), kidney cancer, liver cancer, head and neck cancer, bladder cancer, gastric cancer, gastroesophageal junction cancer, leukaemia, preferably acute myeloid leukaemia or chronic lymphocytic leukaemia, myeloma, preferably multiple myeloma and lymphoma, preferably diffuse large B-cell lymphoma (DLBCL), B-Cell Lymphoma, Follicular Lymphoma, Mantle Cell Lymphoma, Lymphoma of Mucosa-Associated Lymphoid Tissue (MALT), T-Cell/Histiocyte-Rich B-Cell Lymphoma, Burkitt's Lymphoma, Lymphoplasmacytic Lymphoma, Small Lymphocytic Lymphoma, Marginal Zone Lymphoma, T Cell Lymphoma, Peripheral T-Cell Lymphoma, Anaplastic Large Cell Lymphoma and AngioImmunoblastic T-Cell Lymphoma.

Preferably, the cancer is selected from the list comprising colorectal cancer, pancreatic cancer, gastric cancer, gastroesophageal junction cancer, endometrial cancer, bladder cancer, breast cancer, ovarian cancer, esophageal cancer, renal cancer and lung cancer.

In preferred embodiments the patient is a human.

The skilled person will understand that the platin drug can be administered either before, at the same time as, or subsequent to administration of the anti-LY75 antibody, or an antigen-binding portion thereof.

In some embodiments the platin drug is administered, 1 day, 2 days, 3, days, 4 days, 5 days, 6 days, 7 days, 8 days, 9 days, 10 days, 2 weeks, or 3 weeks after administration of the antibody or antigen binding portion thereof which binds to LY75.

In one embodiment the platin drug is administered 2 days or 3 days after administration of the antibody or antigen binding portion thereof which binds to LY75.

Also within the scope of the invention are kits comprising a pharmaceutical combination of the invention and, optionally, instructions for use. The kit can further contain a least one additional reagent or one or more additional antibodies.

Other features and advantages of the instant invention will be apparent from the following detailed description and claims.

### BRIEF DESCRIPTION OF THE DRAWINGS

FIG. 1 depicts the alignment of LY75_A1 heavy chain (SEQ ID NO:1), the human VH 3-15 Germline (SEQ ID NO:11) and the human JH4 Germline (SEQ ID NO:12). The CDR regions of LY75_A1 heavy chain are underlined.

FIG. 2 depicts the alignment of LY75_A1 light chain (SEQ ID NO:2), the human VK O12 Germline (SEQ ID NO:13) and the human JK4 Germline (SEQ ID NO:14). The CDR regions of LY75_A1 light chain are underlined.

FIG. 3a depicts cytotoxic activity of anti-LY75 monoclonal antibodies conjugated with DM1 in HT-29 and shows while most antibodies bind to LY75 only a few display efficacy.

FIG. 3b depicts cytotoxic activity of anti-LY75 antibodies conjugated to either DM1 or DM4 in HT-29.

FIG. 3c depicts cytotoxic activity of anti-LY75 antibodies conjugated to either DM1 or DM4 in RAJI cells.

FIG. 3d depicts cytotoxic activity of anti-LY75 antibodies conjugated to either DM1 or DM4 in Namalwa cells.

FIG. 3e depicts cytotoxic activity of anti-LY75 antibodies conjugated to either DM1 or DM4 in Karpas 299 cells.

FIG. 3f depicts cytotoxic activity of anti-LY75 antibodies conjugated to either DM1 or DM4 in BxPC3 cells.

FIG. 3g depicts cytotoxic activity of anti-LY75 antibodies conjugated to either DM1 or DM4 in HupT4 cells.

FIG. 3h depicts cytotoxic activity of anti-LY75 antibodies conjugated to either DM1 or DM4 in HPAFFII cells.

FIG. 3i depicts cytotoxic activity of anti-LY75 antibodies conjugated to either DM1 or DM4 in EHEB cells.

FIG. 3j depicts cytotoxic activity of anti-LY75 antibodies conjugated to either DM1 or DM4 in Mec-1 cells.

FIG. 3k depicts cytotoxic activity of anti-LY75 antibodies conjugated to either DM1 or DM4 in AML-193 cells.

FIG. 3l depicts cytotoxic activity of anti-LY75 antibodies conjugated to either DM1 or DM4 in HCC 70 cells.

FIG. 3m depicts cytotoxic activity of anti-LY75 antibodies conjugated to either DM1 or DM4 in HCC 1806 cells.

FIG. 3n depicts cytotoxic activity of anti-LY75 antibodies conjugated to either DM1 or DM4 in MDA-MB-468 cells.

FIG. 3o depicts cytotoxic activity of anti-LY75 antibodies conjugated to either DM1 or DM4 in RT4 cells.

FIG. 3p depicts cytotoxic activity of anti-LY75 antibodies conjugated to either DM1 or DM4 in 5637 cells.

FIG. 3q depicts cytotoxic activity of anti-LY75 antibodies conjugated to either DM1 or DM4 in SW780 cells.

FIG. 3r depicts cytotoxic activity of anti-LY75 antibodies conjugated to either DM1 or DM4 in SCC-9 cells.

FIG. 3s depicts cytotoxic activity of anti-LY75 antibodies conjugated to either DM1 or DM4 in OE 19 cells.

FIG. 3t depicts cytotoxic activity of anti-LY75 antibodies conjugated to either DM1 or DM4 in OVCAR-3 cells.

FIG. 3u depicts cytotoxic activity of anti-LY75 antibodies conjugated to either DM1 or DM4 in SK-OV-3 cells.

FIG. 3v depicts cytotoxic activity of anti-LY75 antibodies conjugated to either DM1 or DM4 in MOLP-8 cells.

FIG. 3w depicts cytotoxic activity of anti-LY75 antibodies conjugated to either DM1 or DM4 in RPMI8226 cells.

FIG. 4a depicts the efficacy of anti-LY75 antibodies conjugated to either DM1 or DM4 in Raji Burkitt's lymphoma SCID mouse xenograft model.

FIG. 4b depicts the efficacy of anti-LY75 antibodies conjugated to either DM1 or DM4 in Namalwa Burkitt's lymphoma SCID mouse xenograft model.

FIG. 4c depicts the efficacy of anti-LY75 antibodies conjugated to either DM1 or DM4 in HPAFII pancreatic adenocarcinoma athymic nude mousexenograft model.

FIG. 4d depicts the efficacy of anti-LY75 antibodies conjugated to either DM1 or DM4 in SW780 human bladder carcinoma SCID mouse xenograft model.

FIG. 4e depicts the efficacy of anti-LY75 antibodies conjugated to either DM1 or DM4 in MDA-MB-468 athymic nude mouse xenograft model.

FIG. 4f depicts the efficacy of anti-LY75 antibodies conjugated to either DM1 or DM4 in COLO205 colorectal adenocarcinoma athymic nude mouse xenograft model.

FIG. 5A shows the effect of treatment of HT-29 cells with either oxaliplatin alone or oxaliplatin on cells that have been pre-treated with 0.5 nM or 2 nM anti-LY75_A1 for 72 hrs.

FIG. 5B shows the effect of treatment of HT-29 cells with either cisplatin alone or cisplatin on cells that have been pre-treated with 0.5 nM or 2 nM anti-LY75_A1 for 72 hrs.

FIG. 5C shows the effect of treatment of HPAFII cells with either oxaliplatin alone or oxaliplatin on cells that have been pre-treated with 1 nM, 3 nM or 10 nM anti-LY75_A1 for 72 hrs.

FIG. 5D shows the effect of treatment of N87 cells with either oxaliplatin alone or oxaliplatin on cells that have been pre-treated with 1 nM or 10 nM anti-LY75_A1 for 72 hrs.

FIG. 5E shows the effect of treatment of N87 cells with either cisplatin alone or cisplatin on cells that have been pre-treated with 1 nM or 10 nM anti-LY75_A1 for 72 hrs.

FIG. 6 shows that administration of the combination of 1 mg/kg anti-LY75 and 5 mg/kg of oxaliplatin significantly reduces tumor growth in mice having N87 gastric cancer xenografts.

### DETAILED DESCRIPTION OF THE INVENTION

The present disclosure relates to pharmaceutical combinations comprising an anti-LY75 antibody, or an antigen-binding portion thereof, and a platin drug or a pharmaceutically acceptable salt thereof, wherein the pharmaceutical combination is in the form of a combined preparation for simultaneous, separate or sequential use.

One example of the LY75 protein is given in SEQ ID NO: 15 herein. The terms “anti-LY75 antibodies” and “LY75 antibodies” are used interchangeably herein.

The LY75 antibodies disclosed herein may be internalized when contacted with cells expressing the LY75 receptor. As discussed herein, the LY75 receptor is overexpressed and/or differentially expressed on certain cancer cells, for example, colorectal cancer, breast cancer, bladder cancer, gastric cancer, ovarian cancer, lung cancer, pancreatic cancer, esophageal cancer and renal cancer cells.

As such, when the LY75 antibodies disclosed herein are conjugated to drugs (sometimes referred to herein as “antibody-drug conjugates” or “ADCs”), the internalization of these ADC molecules into cancer cells results in cell death and thus tumor treatment.

The anti-LY75 antibodies possess particular structural features such as CDR regions with particular amino acid sequences. Described herein are a set of CDRs which can form an affinity reagent, e.g. an antibody, which exhibits binding to LY75.

Any of the anti-LY75 antibodies of the invention may be isolated antibodies.

Thus, the disclosure provides antibodies, preferably isolated antibodies (which, as outlined below, includes a wide variety of well-known antibody structures, derivatives, mimetics and conjugates), nucleic acids encoding antibody combinations, host cells used to make the antibody combinations, methods of making the antibody combinations, and pharmaceutical combinations comprising the antibodies and optionally a pharmaceutical carrier, methods of treatment comprising the use of the pharmaceutical combinations and the use of the pharmaceutical combinations for the treatment of cancers.

Expression of LY75 has been observed in pancreatic, bladder, gastric, ovarian, breast (including triple negative), colorectal, esophageal, skin, thyroid, renal, and lung (non-small-cell) cancers as well as Multiple Myeloma and many different subtypes of lymphomas (including DLBCL) and leukaemias.

The anti-LY75 antibody may, in certain cases, cross-react with the LY75 from species other than human. For example, to facilitate clinical testing, the anti-LY75 antibodies may cross react with murine or primate LY75 molecules. Alternatively, in certain embodiments, the antibodies may be completely specific for human LY75 and may not exhibit species or other types of non-human cross-reactivity.

Antibodies that find use in the present invention can take on a number of formats as described herein, including traditional antibodies as well as antibody derivatives, fragments and mimetics, described below. In one embodiment, the invention provides antibody structures that contain a set of 6 CDRs as defined herein (including small numbers of amino acid changes as described below).

“Antibody” as used herein includes a wide variety of structures, as will be appreciated by those in the art, that in some embodiments contain at a minimum a set of 6 CDRs as defined herein; including, but not limited to traditional antibodies (including both monoclonal and polyclonal antibodies), humanized and/or chimeric antibodies, antibody fragments, engineered antibodies (e.g. with amino acid modifications as outlined below), multispecific antibodies (including bispecific antibodies), and other analogs known in the art.

“Isotype” as used herein is means any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. It should be understood that therapeutic antibodies can also comprise hybrids of any combination of isotypes and/or subclasses.

In many embodiments, IgG isotypes are used in the present invention, with IgG1 finding particular use in a number of applications.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. As described herein, methods for determining what epitopes are bound by a given antibody (i.e., epitope mapping) are well known in the art and include, for example, immunoblotting and immunoprecipitation assays, wherein overlapping or contiguous peptides from LY75 are tested for reactivity with the given anti-LY75 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)). The term “epitope mapping” refers to the process of identification of the molecular determinants for antibody-antigen recognition.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al.).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus, for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

Of particular interest in the present invention are the Fc regions. By “Fc” or “Fc region” or “Fc domain” as used herein is meant the polypeptide comprising the constant region of an antibody excluding the first constant region immunoglobulin domain and in some cases, part of the hinge. In some embodiments, as is more fully described below, amino acid modifications are made to the Fc region, for example to alter binding to one or more FcγR receptors or to the FcRn receptor.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Structures that rely on the use of a set of CDRs are included within the definition of “antibody”.

In one embodiment, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable region, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

In some embodiments, the antibody can be a mixture from different species, e.g. a chimeric antibody and/or a humanized antibody. That is, in the present invention, the CDR sets can be used with framework and constant regions other than those specifically described by sequence herein.

In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272 (16): 10678-10684; Rosok et al., 1996, J. Biol. Chem. 271 (37): 22611-22618; Rader et al., 1998, Proc. Natl. Acad. Sci. USA 95:8910⁻⁸⁹¹⁵; Krauss et al., 2003, Protein Engineering 16 (10): 753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

The antibodies disclosed herein may be isolated or recombinant. “Isolated,” when used to describe the various polypeptides disclosed herein, means a polypeptide that has been identified and separated and/or recovered from a cell or cell culture from which it was expressed. Thus, an isolated antibody is intended to refer to an antibody that is substantially free of other antibodies having different antigenic specificities (e.g. an isolated antibody that specifically binds to the LY75 is substantially free of antibodies that specifically bind antigens other than the LY75). Thus, an “isolated” antibody is one found in a form not normally found in nature (e.g. non-naturally occurring). An isolated antibody as defined herein may, in one embodiment, include at least one amino acid which does not occur in the “naturally” occurring antibody. This amino acid may be introduced by way of an addition or a substitution. It will be understood that the introduced amino acid may be a naturally occurring or non-naturally occurring amino acid. In some embodiments, the antibodies of the invention are recombinant proteins, isolated proteins or substantially pure proteins. An “isolated” protein is unaccompanied by at least some of the material with which it is normally associated in its natural state, for example constituting at least about 5%, or at least about 50% by weight of the total protein in a given sample. It is understood that the isolated protein may constitute from 5 to 99.9% by weight of the total protein content depending on the circumstances. For example, the protein may be made at a significantly higher concentration through the use of an inducible promoter or high expression promoter, such that the protein is made at increased concentration levels. In the case of recombinant proteins, the definition includes the production of an antibody in a wide variety of organisms and/or host cells that are known in the art in which it is not naturally produced. Ordinarily, an isolated polypeptide will be prepared by at least one purification step. An “isolated antibody,” refers to an antibody which is substantially free of other antibodies having different antigenic specificities. For instance, an isolated antibody that specifically binds to LY75 is substantially free of antibodies that specifically bind antigens other than LY75. The isolated anti-LY75 antibody may, of course, be associated with one or more platins.

Isolated monoclonal antibodies, having different specificities, can be combined in a well-defined composition. Thus, for example, the antibody of the invention can optionally and individually be included or excluded in a formulation, as is further discussed below.

The anti-LY75 antibodies of the present invention specifically bind LY75 (e.g. SEQ ID NO: 15). “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope. However, in the present invention, when administering ADCs of the LY75 antibodies of the invention, what is important is that the K_{D} is sufficient to allow internalization and thus cell death without significant side effects.

Also, specific binding for a particular antigen or an epitope can be exhibited, for example, by an antibody having a K_{A} or Kₐ for an antigen or epitope of at least 20-, 50-, 100-, 500-, 1000-, 5,000-, 10,000- or more times greater for the epitope relative to a control, where K_{A} or Kₐ refers to an association rate of a particular antibody-antigen interaction.

The LY75 antibodies that bind to LY75 (SEQ ID NO: 15) may be internalized when contacted with cells expressing LY75 on the cell surface. These antibodies are referred to herein either as “anti-LY75” antibodies or, for ease of description, “LY75 antibodies”. Both terms are used interchangeably herein.

LY75 antibodies as defined herein that also comprise drug conjugates are internalized by tumor cells, resulting in the release of the drug and subsequent cell death, allowing for treatment of cancers that exhibit LY75 expression. Internalization in this context can be measured in several ways. In one embodiment, the LY75 antibodies are contacted with cells, such as a cell line as outlined herein, using standard assays such as MAbZap. It would be clear to the skilled person that the MabZap assay is representative of the effect that would be expected to be seen with an antibody-drug conjugate (ADC). In the latter case, the ADC would be internalized, thus taking the drug into the cell. A toxic drug would have the capacity to kill the cell, i.e. to kill the targeted cancer cell. Data from MabZap assays are readily accepted by persons of skill in the art to be representative of ADC assays (Kohls, M and Lappi, D., Biotechniques, vol. 28, no. 1, 162-165).

In these in vitro assay embodiments, the LY75 antibodies are added, along with an anti-LY75 antibody comprising a toxin; for example, the LY75 antibody may be murine or humanized and the anti-LY75 antibody can be anti-murine or anti-humanized and contain a toxin such as saporin. Upon formation of the [LY75 antibody]-[anti-LY75 antibody-drug conjugate] complex, the complex is internalized and the drug (e.g. saporin) is released, resulting in cell death. Only upon internalization does the drug get released, and thus cells remain viable in the absence of internalization. As outlined below, without being bound by theory, in therapeutic applications, the anti-LY75 antibody contains the toxin, and upon internalization, the bond between the antibody and the toxin is cleaved, releasing the toxin and killing the cell.

In one embodiment, the anti-LY75 antibody comprises the heavy and light chain complementarity determining regions (CDRs) or variable regions (VRs) of the particular antibody described herein (e.g., referred to herein as “LY75_A1”). Accordingly, in one embodiment, the antibody comprises the CDR1, CDR2, and CDR3 domains of the heavy chain variable (VH) region of antibody LY75_A1 having the sequence shown in SEQ ID NO:1, and the CDR1, CDR2 and CDR3 domains of the light chain variable (VL) region of antibody LY75_A1 having the sequence shown in SEQ ID NO:2.

In another embodiment, the anti-LY75 antibody comprises a heavy chain variable region comprising a first vhCDR comprising SEQ ID NO: 5; a second vhCDR comprising SEQ ID NO: 6; and a third vhCDR comprising SEQ ID NO:7; and a light chain variable region comprising a first vICDR comprising SEQ ID NO:8; a second vICDR comprising SEQ ID NO: 9; and a third vICDR comprising SEQ ID NO:10.

In another embodiment, the anti-LY75 antibodies bind to human LY75 and include a heavy chain variable region comprising an amino acid sequence comprising SEQ ID NO:1, and conservative sequence modifications thereof. The antibody may further include a light chain variable region comprising an amino acid sequence comprising SEQ ID NO:2, and conservative sequence modifications thereof.

In a further embodiment, the anti-LY75 antibodies bind to human LY75 and include a heavy chain variable region and a light chain variable region comprising the amino acid sequences set forth in SEQ ID NOs: 1 and/or 2, respectively, and conservative sequence modifications thereof.

In a further embodiment, the anti-LY75 antibodies bind to human LY75 and include a heavy chain and a light chain comprising the amino acid sequences set forth in SEQ ID NOs: 24 and/or 25, respectively, and conservative sequence modifications thereof.

As used herein, the term conservative sequence modification refers to, for example, the substitution of an amino acid with an amino acid having similar characteristics. It is common general knowledge for one skilled in the art what such substitutions may be considered conservative. Other modifications which can be considered to be conservative sequence modifications include, for example, glycosylation.

Optionally, one or more of SEQ ID NOs: 5-10 independently comprise one, two, three, four or five conservative amino acid substitutions; optionally, one or more of SEQ ID NOs: 5-10 independently comprise one or two conservative amino acid substitutions.

Preferably, the term “conservative sequence modifications” is intended to include amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function using the functional assays described herein.

Isolated antibodies which include heavy and light chain variable regions having at least 80%, or at least 85%, or at least 90%, or at least 91%, or at least 92%, or at least 93%, or at least 94%, or at least 95%, or at least 96%, or at least 97%, or at least 98%, or at least 99%, or more sequence identity to any of the above sequences are also included in the present invention. Ranges intermediate to the above-recited values, e.g., heavy and light chain variable regions having at least 80-85%, 85-90%, 90-95% or 95-100% sequence identity to any of the above sequences are also intended to be encompassed by the present invention. In one embodiment, the anti-LY75 antibody comprises a heavy chain variable region comprising SEQ ID NO:1 or a sequence that is at least 90%, at least 91%, at least 92%, at least 93%, at least 94%, at least 95%, at least 96%, at least 97%, at least 98%, or at least 99% identical to SEQ ID NO: 1. In another embodiment, the anti-LY75 antibody comprises a light chain variable region comprising SEQ ID NO:2 or a sequence that is at least 90%, at least 91%, at least 92%, at least 93%, at least 94%, at least 95%, at least 96%, at least 97%, at least 98%, or at least 99% identical to SEQ ID NO: 2. In another embodiment, the anti-LY75 antibody comprises a heavy chain framework region comprising an amino acid sequence that is at least 90%, at least 91%, at least 92%, at least 93%, at least 94%, at least 95%, at least 96%, at least 97%, at least 98%, or at least 99% identical to the framework of the heavy chain variable region of SEQ ID NO: 1 comprising SEQ ID NOs: 16, 17 and 18. In another embodiment, the anti-LY75 antibody comprises a light chain framework region comprising an amino acid sequence that is at least 90%, at least 91%, at least 92%, at least 93%, at least 94%, at least 95%, at least 96%, at least 97%, at least 98%, or at least 99% identical to the framework of the light chain variable region of SEQ ID NO:2 comprising SEQ ID NOs: 19, 20 and 21.

In a further embodiment, the anti-LY75 antibody comprises a heavy chain comprising SEQ ID NO: 24 or a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 24. In another embodiment, the anti-LY75 antibody comprises a light chain comprising SEQ ID NO: 25 or a sequence that is at least 80%, at least 85%, at least 90%, at least 91%, at least 92%, at least 93%, at least 94%, at least 95%, at least 96%, at least 97%, at least 98%, or at least 99% identical to SEQ ID NO: 25. The heavy chain may comprise the sequences of SEQ ID NOs: 5-7 or 1. The light chain may comprise the sequences of SEQ ID NOs: 8-10 or 2.

In one embodiment, the anti-LY75 antibody is referred to herein as “LY75_A1 antibody” comprising the following CDRs, as well as variants containing a limited number of amino acid variants:

| A1 | SEQ ID NOS |
|---|---|
| variable heavy CDR1 | 5 |
| variable heavy CDR2 | 6 |
| variable heavy CDR3 | 7 |
| variable light CDR1 | 8 |
| variable light CDR2 | 9 |
| variable light CDR3 | 10 |

Disclosed herein are also variable heavy and light chains that comprise the CDR sets of the invention, as well as full length heavy and light chains (e.g. comprising constant regions as well). As will be appreciated by those in the art, the CDR sets of the anti-LY75 antibody can be incorporated into murine, humanized or human constant regions (including framework regions). Accordingly, the present disclosure provides variable heavy and light chains and full length heavy and light chains that are at least about 90%-99% identical to the SEQ IDs disclosed herein, with 90, 91, 92, 93, 94, 95, 96, 97, 98 and 99% all finding use in the present invention.

In some embodiments, the anti-LY75 antibody is one which competes for binding to human LY75 with an antibody comprising a heavy chain variable region comprising the amino acid sequence set forth in SEQ ID NO: 1, and a light chain variable region comprising the amino acid sequence set forth in SEQ ID NO: 2. Antibodies that compete for binding can be identified using routine techniques. Such techniques include, for example, an immunoassay, which shows the ability of one antibody to block the binding of another antibody to a target antigen, i.e., a competitive binding assay. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% 75-80% 80-85% 85-90% 90-95% 95-99% or more.

Monoclonal antibodies can be characterized for binding to LY75 using a variety of known techniques. Generally, the antibodies are initially characterized by ELISA

An ELISA assay can be used to screen for antibodies and, thus, hybridomas that produce antibodies that show positive reactivity with the LY75 immunogen. Hybridomas that bind, preferably with high affinity, to LY75 can then be sub-cloned and further characterized. One clone from each hybridoma, which retains the reactivity of the parent cells (by ELISA), can then be chosen for making a cell bank, and for antibody purification.

To purify anti-LY75 antibodies, selected hybridomas can be grown in roller bottles, two-liter spinner-flasks or other culture systems. Supernatants can be filtered and concentrated before affinity chromatography with protein A-Sepharose (Pharmacia, Piscataway, NJ) to purify the protein. After buffer exchange to PBS, the concentration can be determined by OD₂₈₀ using 1.43 extinction coefficient or preferably by nephelometric analysis. IgG can be checked by gel electrophoresis and by antigen specific method.

To determine if the selected anti-LY75 monoclonal antibodies bind to unique epitopes, each antibody can be biotinylated using commercially available reagents (Pierce, Rockford, IL). Biotinylated MAb binding can be detected with a streptavidin labeled probe. To determine the isotype of purified antibodies, isotype ELISAs can be performed using art recognized techniques.

To test the binding of monoclonal antibodies to live cells expressing LY75, flow cytometry can be used. This method allows visualization of individual cells, but may have diminished sensitivity depending on the density of the antigen.

Anti-LY75 IgGs can be further tested for reactivity with the LY75 antigen by Western blotting.

Methods for analyzing binding affinity, cross-reactivity, and binding kinetics of various anti-LY75 antibodies include standard assays known in the art, for example, Biacore™ surface plasmon resonance (SPR) analysis using a Biacore™ 2000 SPR instrument (Biacore AB, Uppsala, Sweden.

In one embodiment, the antibody specifically binds to human LY75 comprising SEQ ID NO: 15). Preferably, the anti-LY75 antibody binds to human LY75 with high affinity. Preferably, the anti-LY75 antibody binds to a LY75 protein with a K_{D} of 5×10⁻⁸ M or less, binds to a LY75 protein with a K_{D} of 2×10⁻⁸ M or less, binds to a LY75 protein with a K_{D} of 5×10⁻⁹ M or less, binds to a LY75 protein with a K_{D} of 4×10⁻⁹ M or less, binds to a LY75 protein with a K_{D} of 3×10⁻⁹ M or less, binds to a LY75 protein with a K_{D} of 2×10⁻⁹ M or less, binds to a LY75 protein with a K_{D} of 1×10⁻⁹ M or less, binds to a LY75 protein with a K_{D} of 5×10⁻¹⁰ M or less, or binds to a LY75 protein with a K_{D} of 1×10⁻¹⁰ M or less.

The present invention encompasses variant antibodies, sometimes referred to as “antibody derivatives” or “antibody analogs” as well. That is, there are a number of modifications that can be made to the antibodies disclosed herein, including, but not limited to, amino acid modifications in the CDRs (affinity maturation), amino acid modifications in the framework regions, amino acid modifications in the Fc region, glycosylation variants, covalent modifications of other types (e.g. for attachment of drug conjugates, etc.).

By “variant” herein is meant a polypeptide sequence that differs from that of a parent polypeptide by virtue of at least one amino acid modification. In this case, the parent polypeptide is either the full length variable heavy or light chains, e.g. as listed in SEQ ID NOs: 1 or 2, respectively or the CDR regions or the framework regions of the heavy and light chains listed in SEQ ID NOs 5-10 and 16-21 for LY75. Amino acid modifications can include substitutions, insertions and deletions, with the former being preferred in many cases. It will be understood that an amino acid substitution may be a conservative or non-conservative substitution with conservative substitutions being preferred. Further said substitution may be a substitution with either a naturally or non-naturally occurring amino acid.

In general, variants can include any number of modifications, as long as the function of the antibody is still present, as described herein. That is, LY75_A1, for example, the antibody should still specifically bind to human LY75. If amino acid variants are generated with the Fc region, for example, the variant antibodies should maintain the required receptor binding functions for the particular application or indication of the antibody.

“Variants” in this case can be made in the listed CDR sequences, the framework or Fc regions of the antibody.

However, in general, from 1, 2, 3, 4, 5, 6, 7, 8, 9 or 10 amino acid substitutions are generally utilized as often the goal is to alter function with a minimal number of modifications. In some cases, there are from 1 to 5 modifications (e.g. individual amino acid substitutions, insertions or deletions), with from 1-2, 1-3 and 1-4 also finding use in many embodiments. The number of modifications can depend on the size of the region being modified; for example, in general, fewer modifications are desired in CDR regions. It will be understood by the skilled person that even within the CDR regions the location of the modification can significantly alter the effect. In one embodiment, the modifications can be made in any of CDR1, CDR2 or CDR3 of the heavy and/or light chains. In a further embodiment, the modifications are made in any of CDR1 or CDR2 of the heavy and/or light chains. In a still further embodiment, the modifications are located in CDR1 of the heavy and/or light chains.

It should be noted that the number of amino acid modifications may be within functional domains: for example, it may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 91%, 92%, 93%, 94%, 95%, 96%, 97%, 98% or 99% identity to the parent sequences (e.g. the variable regions, the constant regions, and/or the heavy and light chain sequences and/or the CDRs of LY75_A1). It should be noted that depending on the size of the sequence, the percent identity will depend on the number of amino acids.

By “amino acid substitution” or “substitution” herein is meant the replacement of an amino acid at a particular position in a parent polypeptide sequence with another amino acid which may be a natural or non-naturally occurring amino acid. For example, the substitution S100A refers to a variant polypeptide in which the serine at position 100 is replaced with alanine. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

By “parent polypeptide”, “parent protein”, “precursor polypeptide”, or “precursor protein” as used herein is meant an unmodified polypeptide that is subsequently modified to generate a variant. In general, the parent polypeptide herein is LY75_A1. Accordingly, by “parent antibody” as used herein is meant an antibody that is modified to generate a variant antibody.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

By “variant Fc region” herein is meant an Fc sequence that differs from that of a wild-type Fc sequence by virtue of at least one amino acid modification. Fc variant may refer to the Fc polypeptide itself, compositions comprising the Fc variant polypeptide, or the amino acid sequence.

In some embodiments, one or more amino acid modifications are made in one or more of the CDRs of LY75_A1. In general, only 1 or 2 or 3 amino acids are substituted in any single CDR, and generally no more than from 4, 5, 6, 7, 8 9 or 10 changes are made within a set of 6 CDRs. However, it should be appreciated that any combination of no substitutions, 1, 2 or 3 substitutions in any CDR can be independently and optionally combined with any other substitution. It will be apparent that substitutions can be made in any of the 6 CDRs. In one embodiment, substitutions are made in CDR1 of the heavy and/or light chains.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies disclosed herein are variant CDRs and antibodies; that is, the antibodies can include amino acid modifications in one or more of the CDRs of LY75_A1. In addition, amino acid modifications can also independently and optionally be made in any region outside the CDRs, including framework and constant regions as described herein.

In some embodiments, the anti-LY75 antibodies disclosed herein are composed of a variant Fc domain. As is known in the art, the Fc region of an antibody interacts with a number of Fc receptors and ligands, imparting an array of important functional capabilities referred to as effector functions. These Fc receptors include, but are not limited to, (in humans) FcγRI (CD64) including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158, correlated to antibody-dependent cell cytotoxicity (ADCC)) and FcγRIIIb (including allotypes FcγRIIIb-NA1 and FcγRIIIb-NA2), FcRn (the neonatal receptor), C1q (complement protein involved in complement dependent cytotoxicity (CDC)) and FcRn (the neonatal receptor involved in serum half-life). Suitable modifications can be made at one or more positions.

In addition to the modifications outlined above, other modifications can be made. For example, the molecules may be stabilized by the incorporation of disulphide bridges linking the VH and VL domains (Reiter et al., 1996, Nature Biotech. 14:1239-1245, entirely incorporated by reference).

In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

In addition, as will be appreciated by those in the art, labels (including fluorescent, enzymatic, magnetic, radioactive, etc. can all be added to the antibodies (as well as the other compositions of the invention).

Another type of covalent modification is alterations in glycosylation. In some embodiments, the antibodies disclosed herein can be fully or partially aglycosylated, e.g. afucosylated.

Another type of covalent modification of the antibody comprises linking the antibody to various non-proteinaceous polymers, including, but not limited to, various polyols such as polyethylene glycol, polypropylene glycol or polyoxyalkylenes. In addition, as is known in the art, amino acid substitutions may be made in various positions within the antibody to facilitate the addition of polymers such as PEG. See for example, U.S. Publication No. 2005/0114037A1, entirely incorporated by reference.

In additional embodiments, the antibodies may comprise a label. By “labeled” herein is meant that a compound has at least one element, isotope or chemical compound attached to enable the detection of the compound. In general, labels fall into three classes: a) isotopic labels, which may be radioactive or heavy isotopes; b) magnetic, electrical, thermal; and c) colored or luminescent dyes; although labels include enzymes and particles such as magnetic particles as well. Preferred labels include, but are not limited to, fluorescent lanthanide complexes (including those of Europium and Terbium), and fluorescent labels including, but not limited to, quantum dots, fluorescein, rhodamine, tetramethylrhodamine, eosin, erythrosin, coumarin, methyl-coumarins, pyrene, Malacite green, stilbene, Lucifer Yellow, Cascade Blue, Texas Red, the Alexa dyes, the Cy dyes, and others described in the 6th Edition of the Molecular Probes Handbook by Richard P. Haugland, hereby expressly incorporated by reference.

### Antibody-Drug Conjugates

In some embodiments, the anti-LY75 antibodies disclosed herein are conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc. An overview of this technology is provided in Ducry et al., Bioconjugate Chem., 21:5-13 (2010), Carter et al., Cancer J. 14 (3): 154 (2008) and Senter, Current Opin. Chem. Biol. 13:235-244 (2009), all of which are hereby incorporated by reference in their entirety.

Thus the invention provides pharmaceutical combinations comprising, inter alia, anti-LY75 antibodies conjugated to drugs. Generally, conjugation is done by covalent attachment to the antibody, as further described below, and generally relies on a linker, often a peptide linkage (which, as described below, may be designed to be sensitive to cleavage by proteases at the target site or not). In addition, as described above, linkage of the linker-drug unit (LU-D) can be done by attachment to cysteines within the antibody. As will be appreciated by those in the art, the number of drug moieties per antibody can change, depending on the conditions of the reaction, and can vary from 1:1 to 10:1 drug: antibody. As will be appreciated by those in the art, the actual number is an average.

Thus the anti-LY75 antibodies may be conjugated to drugs. As described below, the drug of the ADC can be any number of agents, including but not limited to cytotoxic agents such as chemotherapeutic agents, growth inhibitory agents, toxins (for example, an enzymatically active toxin of bacterial, fungal, plant, or animal origin, or fragments thereof), or a radioactive isotope (that is, a radioconjugate) are provided. In other embodiments, the invention further provides methods of using the ADCs.

Drugs for use in the present invention include cytotoxic drugs, particularly those which are used for cancer therapy. Such drugs include, in general, DNA damaging agents, anti-metabolites, natural products and their analogs. Exemplary classes of cytotoxic agents include the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, dolastatins, maytansinoids, differentiation inducers, and taxols.

Members of these classes include, for example, taxol, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, hemiasterlins, maytansinoids (including DM1 or DM4), monomethylauristatin E (MMAE), monomethylauristatin F (MMAF), and their analogues.

Toxins may be used as antibody-toxin conjugates and include bacterial toxins such as diphtheria toxin, plant toxins such as ricin, small molecule toxins such as geldanamycin (Mandler et al (2000) J. Nat. Cancer Inst. 92 (19): 1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342), hemiasterlins (WO2004/026293; Zask et al., (2004) J. Med. Chem, 47:4774-4786). Toxins may exert their cytotoxic and cytostatic effects by mechanisms including tubulin binding, DNA binding, or topoisomerase inhibition.

Conjugates of an anti-LY75 antibody and one or more small molecule toxins, such as a maytansinoids, dolastatins, auristatins, a trichothecene, calicheamicin, duocarmycins, pyrrolobenzadiazepines and CC1065, and the derivatives of these toxins that have toxin activity, may also be used.

Preferably, the anti-LY75 antibody is conjugated to DM1 or DM4, most preferably to DM4. Maytansine compounds suitable for use as maytansinoid drug moieties are well known in the art, and can be isolated from natural sources according to known methods, produced using genetic engineering techniques (see Yu et al (2002) PNAS 99:7968-7973), or maytansinol and maytansinol analogues prepared synthetically according to known methods. As described below, drugs may be modified by the incorporation of a functionally active group such as a thiol or amine group for conjugation to the antibody.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides) and those having modifications at other positions.

Exemplary maytansinoid drug moieties also include those having modifications such as: C-9-SH (U.S. Pat. No. 4,424,219) (prepared by the reaction of maytansinol with H2S or P2S5); C-14-alkoxymethyl (demethoxy/CH₂OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Of particular use are DM1 (disclosed in U.S. Pat. No. 5,208,020, incorporated by reference) and DM4 (disclosed in U.S. Pat. No. 7,276,497, incorporated by reference). See also a number of additional maytansinoid derivatives and methods in 5,416,064, WO/01/24763, 7,303,749, 7,601,354, U.S. Ser. No. 12/631,508, WO02/098883, 6,441,163, 7,368,565, WO02/16368 and WO04/1033272, all of which are expressly incorporated by reference in their entirety.

ADCs containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference. Liu et al., Proc. Natl. Acad. Sci. USA 93:8618-8623 (1996) described ADCs comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay.

Chari et al., Cancer Research 52:127-131 (1992) describe ADCs in which a maytansinoid was conjugated via a disulfide linker to the murine antibody A7 binding to an antigen on human colon cancer cell lines, or to another murine monoclonal antibody TA. 1 that binds the HER-2/neu oncogene. The cytotoxicity of the TA. 1-maytansonoid conjugate was tested in vitro on the human breast cancer cell line SK-BR-3, which expresses 3×105 HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

For compositions comprising a plurality of antibodies, the drug loading is represented by p, the average number of drug molecules per Antibody. Drug loading may range from 1 to 20 drugs (D) per Antibody. The average number of drugs per antibody in preparation of conjugation reactions may be characterized by conventional means such as mass spectroscopy, ELISA assay, and HPLC. The quantitative distribution of Antibody-Drug-Conjugates in terms of p may also be determined.

In some instances, separation, purification, and characterization of homogeneous Antibody-Drug-conjugates where p is a certain value from Antibody-Drug-Conjugates with other drug loadings may be achieved by means such as reverse phase HPLC or electrophoresis. In exemplary embodiments, p is 2, 3, 4, 5, 6, 7, or 8 or a fraction thereof.

The generation of Antibody-drug conjugate compounds can be accomplished by any technique known to the skilled artisan. Briefly, the Antibody-drug conjugate compounds can include an anti-LY75 antibody as the Antibody unit, a drug, and optionally a linker that joins the drug and the binding agent.

A number of different reactions are available for covalent attachment of drugs and/or linkers to binding agents. This is can be accomplished by reaction of the amino acid residues of the binding agent, for example, antibody molecule, including the amine groups of lysine, the free carboxylic acid groups of glutamic and aspartic acid, the sulfhydryl groups of cysteine and the various moieties of the aromatic amino acids. A commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule.

Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates can also be used as coupling agents for covalently attaching drugs to binding agents. Other techniques are known to the skilled artisan and within the scope of the present invention.

In some embodiments, an intermediate, which is the precursor of the linker, is reacted with the drug under appropriate conditions. In other embodiments, reactive groups are used on the drug and/or the intermediate. The product of the reaction between the drug and the intermediate, or the derivatized drug, is subsequently reacted with an anti-LY75 antibody of the invention under appropriate conditions.

It will be understood that chemical modifications may also be made to the desired compound in order to make reactions of that compound more convenient for purposes of preparing conjugates of the invention. For example a functional group e.g. amine, hydroxyl, or sulfhydryl, may be appended to the drug at a position which has minimal or an acceptable effect on the activity or other properties of the drug.

Typically, the antibody-drug conjugate compounds comprise a linker unit between the drug unit and the antibody unit. In some embodiments, the linker is cleavable under intracellular or extracellular conditions, such that cleavage of the linker releases the drug unit from the antibody in the appropriate environment. For example, solid tumors that secrete certain proteases may serve as the target of the cleavable linker; in other embodiments, it is the intracellular proteases that are utilized. In yet other embodiments, the linker unit is not cleavable and the drug is released, for example, by antibody degradation in lysosomes.

In some embodiments, the linker is cleavable by a cleaving agent that is present in the intracellular environment (for example, within a lysosome or endosome or caveolea). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long or more.

Cleaving agents can include, without limitation, cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Peptidyl linkers may be cleavable by enzymes that are present in LY75-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO: 46)). Other examples of such linkers are described, e.g., in U.S. Pat. No. 6,214,345, incorporated herein by reference in its entirety and for all purposes.

In some embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker).

In other embodiments, the cleavable linker is pH-sensitive, that is, sensitive to hydrolysis at certain pH values. Typically, the pH-sensitive linker is hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) may be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (for example, a disulfide linker). A variety of disulfide linkers are known in the art, including, for example, those that can be formed using SATA (N-succinimidyl-5-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene)-, SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935).

In other embodiments, the linker is a malonate linker (Johnson et al., 1995, Anticancer Res. 15:1387-93), a maleimidobenzoyl linker (Lau et al., 1995, Bioorg-Med-Chem. 3 (10): 1299-1304), or a 3′-N-amide analog (Lau et al., 1995, Bioorg-Med-Chem. 3 (10): 1305-12).

In yet other embodiments, the linker unit is not cleavable and the drug is released by antibody degradation. (See U.S. Publication No. 2005/0238649 incorporated by reference herein in its entirety and for all purposes).

In many embodiments, the linker is self-immolative. As used herein, the term “self-immolative Spacer” refers to a bifunctional chemical moiety that is capable of covalently linking together two spaced chemical moieties into a stable tripartite molecule. It will spontaneously separate from the second chemical moiety if its bond to the first moiety is cleaved. See for example, WO 2007/059404A2, WO06/110476A2, WO05/112919A2, WO2010/062171, WO09/017394, WO07/089149, WO 07/018431, WO04/043493 and WO02/083180, which are directed to drug-cleavable substrate conjugates where the drug and cleavable substrate are optionally linked through a self-immolative linker and which are all expressly incorporated by reference.

Often the linker is not substantially sensitive to the extracellular environment. As used herein, “not substantially sensitive to the extracellular environment,” in the context of a linker, means that no more than about 20%, 15%, 10%, 5%, 3%, or no more than about 1% of the linkers, in a sample of antibody-drug conjugate compound, are cleaved when the antibody-drug conjugate compound presents in an extracellular environment (for example, in plasma).

Whether a linker is not substantially sensitive to the extracellular environment can be determined, for example, by incubating with plasma the antibody-drug conjugate compound for a predetermined time period (for example, 2, 4, 8, 16, or 24 hours) and then quantitating the amount of free drug present in the plasma.

In other, non-mutually exclusive embodiments, the linker promotes cellular internalization. In certain embodiments, the linker promotes cellular internalization when conjugated to the therapeutic agent (that is, in the milieu of the linker-therapeutic agent moiety of the antibody-drug conjugate compound as described herein). In yet other embodiments, the linker promotes cellular internalization when conjugated to both the auristatin compound and the anti-LY75 antibodies of the invention.

A variety of exemplary linkers that can be used with the present compositions and methods are described in WO 2004/010957, U.S. Publication No. 2006/0074008, U.S. Publication No. 20050238649, and U.S. Publication No. 2006/0024317 (each of which is incorporated by reference herein in its entirety and for all purposes). Preferably, the linker is SPDB (N-succinimidyl-3-(2-pyridyldithio) butyrate).

Drug loading is represented by p and is the average number of Drug moieties per antibody in a molecule. Drug loading (“p”) may be 1, 2, 3, 4, 5, 6, 7, 8, 9, 10, 11, 12, 13, 14, 15, 16, 17, 18, 19, 20 or more moieties (D) per antibody, although frequently the average number is a fraction or a decimal. Generally, drug loading of from 1 to 4 is frequently useful, and from 1 to 2 is also useful. ADCs of the invention include collections of antibodies conjugated with a range of drug moieties, from 1 to 20, for example, 1-15, 1-10, 2-9, 3-8, 4-7, 5-6. The average number of drug moieties per antibody in preparations of ADC from conjugation reactions may be characterized by conventional means such as mass spectroscopy and, ELISA assay.

The quantitative distribution of ADC in terms of p may also be determined. In some instances, separation, purification, and characterization of homogeneous ADC where p is a certain value from ADC with other drug loadings may be achieved by means such as electrophoresis.

For some antibody-drug conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. In certain embodiments, higher drug loading, e.g. p>5, may cause aggregation, insolubility, toxicity, or loss of cellular permeability of certain antibody-drug conjugates. In certain embodiments, the drug loading for an ADC of the invention ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7. Indeed, it has been shown that for certain ADCs, the optimal ratio of drug moieties per antibody may be less than 8, and may be about 2 to about 5. See US 2005/0238649 A1 (herein incorporated by reference in its entirety).

In certain embodiments, less than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug moiety reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography.

In some embodiments, a homogeneous ADC with a single loading value may be isolated from the conjugation mixture by electrophoresis or chromatography.

### Methods of Determining Cytotoxic Effect of ADCs

Methods of determining whether a Drug or Antibody-Drug conjugate exerts a cytostatic and/or cytotoxic effect on a cell are known. Generally, the cytotoxic or cytostatic activity of an Antibody Drug conjugate can be measured by: exposing mammalian cells expressing a target protein of the Antibody Drug conjugate in a cell culture medium; culturing the cells for a period from about 6 hours to about 5 days; and measuring cell viability. Cell-based in vitro assays can be used to measure viability (proliferation), cytotoxicity, and induction of apoptosis (caspase activation) of the Antibody Drug conjugate.

For determining whether an Antibody Drug conjugate exerts a cytostatic effect, a thymidine incorporation assay may be used. For example, cancer cells expressing a target antigen at a density of 5,000 cells/well of a 96-well plated can be cultured for a 72-hour period and exposed to 0.5 μCi of ³H-thymidine during the final 8 hours of the 72-hour period. The incorporation of ³H-thymidine into cells of the culture is measured in the presence and absence of the Antibody Drug conjugate.

For determining cytotoxicity, necrosis or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that an Antibody Drug conjugate is useful in the treatment of cancers.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Apoptosis can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., 1995, Cancer Research 55:3110-16).

In vivo, the effect of a therapeutic composition of the anti-LY75 antibody of the invention can be evaluated in a suitable animal model. For example, xenogenic cancer models can be used, wherein cancer explants or passaged xenograft tissues are introduced into immune compromised animals, such as nude or SCID mice (Klein et al., 1997, Nature Medicine 3:402-408). Efficacy can be measured using assays that measure inhibition of tumor formation, tumor regression or metastasis, and the like.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980).

### Methods for Producing Antibodies

The antibodies disclosed herein may be made by any suitable method. These methods include culturing a host cell containing isolated nucleic acid(s) encoding the antibodies. As will be appreciated by those in the art, this can be done in a variety of ways, depending on the nature of the antibody. In the case where the antibodies are full length traditional antibodies, for example, a heavy chain variable region and a light chain variable region under conditions such that an antibody is produced and can be isolated.

The variable heavy and light chains of LY75_A1 are disclosed herein (both protein and nucleic acid sequences); as will be appreciated in the art, these can be easily augmented to produce full length heavy and light chains. That is, having provided the DNA fragments encoding VH and VK segments as outlined herein, these DNA fragments can be further manipulated by standard recombinant DNA techniques, for example, to convert the variable region genes to full-length antibody chain genes, to Fab fragment genes, or to a scFv gene. In these manipulations, a VK- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H}1, C_{H}2 and C_{H}3). The sequences of murine heavy chain constant region genes are known in the art [see e.g. Kabat, E. A., et al. (1991) Sequences of Proteins of Immunological Interest, Fifth Edition, US Department of Health and Human Services, NIH Publication No. 91-3242] and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H}1 constant region.

The isolated DNA encoding the VL/VK region can be converted to a full-length light chain gene (as well as a Fab light chain gene) by operatively linking the VL-encoding DNA to another DNA molecule encoding the light chain constant region, C_{L}. The sequences of murine light chain constant region genes are known in the art [see, e.g. Kabat, E. A., et al. (1991) Sequences of Proteins of Immunological Interest, Fifth Edition, US Department of Health and Human Services, NIH Publication No. 91-3242] and DNA fragments encompassing these regions can be obtained by standard PCR amplification. In preferred embodiments, the light chain constant region can be a kappa or lambda constant region.

To create a scFv gene, the V_{H}- and V_{L}/V_{K}-encoding DNA fragments are operatively linked to another fragment encoding a flexible linker, e.g. encoding the amino acid sequence (Gly₄-Ser)₃, such that the V_{H} and V_{L}/V_{K} sequences can be expressed as a contiguous single-chain protein, with the V_{L}/V_{K} and V_{H} regions joined by the flexible linker [see e.g. Bird et al. (1988) Science 242:423-426; Huston et al. (1988) Proc. Natl. Acad. Sci. USA 85:5879-5883; McCafferty et al., (1990) Nature 348:552-554].

Nucleic acids are provided which encode the antibodies disclosed herein. Such polynucleotides encode both the variable and constant regions of each of the heavy and light chains, although other combinations are also contemplated in accordance with the compositions described herein.

The polynucleotides can be in the form of RNA or DNA. Polynucleotides in the form of DNA, cDNA, genomic DNA, nucleic acid analogs, and synthetic DNA are also usable. The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

In some embodiments, nucleic acid(s) encoding the antibodies disclosed herein are incorporated into expression vectors, which can be extra-chromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, etc.) or other components (selection genes, etc.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, etc.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, etc.), whereby the encoded polypeptide(s) are produced. In some cases, the heavy chains are produced in one cell and the light chain in another.

Mammalian cell lines available as hosts for expression are known in the art and include many immortalized cell lines available from the American Type Culture Collection (ATCC), Manassas, VA including but not limited to Chinese hamster ovary (CHO) cells, HEK 293 cells, NSO cells, Hela cells, baby hamster kidney (BHK) cells, monkey kidney cells (COS), human hepatocellular carcinoma cells (e.g., Hep G2), and a number of other cell lines. Non-mammalian cells including but not limited to bacterial, yeast, insect, and plants can also be used to express recombinant antibodies. In some embodiments, the antibodies can be produced in transgenic animals such as cows or chickens.

General methods for antibody molecular biology, expression, purification, and screening are well known, for example, see U.S. Pat. Nos. 4,816,567, 4,816,397, 6,331,415 and 7,923,221, as well as Antibody Engineering, edited by Kontermann & Dubel, Springer, Heidelberg, 2001 and 2010 Hayhurst & Georgiou, 2001, Curr Opin Chem Biol 5:683-689; Maynard & Georgiou, 2000, Annu Rev Biomed Eng 2:339-76; and Morrison, S. (1985) Science 229:1202.

The pharmaceutical combination comprises a platin or a pharmaceutically acceptable salt thereof. Platins have been use in the treatment of cancer for a number of years and are well known to those skilled in the art. Platins that may be used in the present invention include, but are not limited to, cisplatin, carboplatin, oxaliplatin nedaplatin, lobaplatin and heptaplatin.

### Pharmaceutical Compositions

The pharmaceutical combination of the invention is in the form of a combined preparation preferably for simultaneous, separate or sequential use. Similarly, in the methods of the invention, the pharmaceutical combination may be administered to a patient simultaneously, separately or sequentially.

The term “combined preparation” or “combination” includes both fixed combinations and non-fixed combinations.

The term “fixed combination” means that the active ingredients are in the form of a single entity or dosage. In other words, the active ingredients are present in a single composition or formulation.

The term “non-fixed combination” means that the active ingredients are present in different entities or dosages (e.g. as separate compositions or formulations), for example as a kit of parts. The independent components (in their desired compositions or formulations) can then be administered separately or sequentially, at the same time point or at different time points.

Where the administration is sequential, the delay in administering the second component should not be such as to lose the benefit of the effect arising from use of the combination. Therefore, in one embodiment sequential treatment involves administration of each component of the combination within a period of 11 days. In another embodiment this period is 10 days. In another embodiment this period is 9 days. In another embodiment this period is 8 days. In another embodiment this period is 7 days. In another embodiment this period is within 6 days. In another embodiment this period is within 5 days. In another embodiment this period is within 4 days. In another embodiment this period is within 3 days. In another embodiment this period is within 2 days. In another embodiment this period is within 24 hours. In another embodiment this period is within 12 hours.

The components of the pharmaceutical combination of the invention may be administered in any order, e.g. the antibody or antigen-binding portion thereof first and then the platin drug; or vice versa.

The ratio of the total amounts of the components to be administered in the combined preparation can be varied, e.g. in order to cope with the needs of a patient sub-population to be treated or the needs of the single patient which different needs can be due to age, sex, body weight, etc. of the patients.

The components of the present invention, whether present in a single composition or in separate compositions, may independently be formulated with one or more pharmaceutically acceptable carriers. The pharmaceutical combinations of the invention may also include at least one other anti-tumor agent, or an anti-inflammatory or immunosuppressant agent. Examples of therapeutic agents that can be used in combination therapy are described in greater detail below in the section on uses of the antibodies disclosed herein.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion). Depending on the route of administration, the active compound, i.e. antibody, immunoconjugate, or bispecific molecule, may be coated in a material to protect the compound from the action of acids and other natural conditions that may inactivate the compound.

The components of the present invention may be in the form of one or more pharmaceutically acceptable salts. A “pharmaceutically acceptable salt” refers to a salt that retains the desired biological activity of the parent compound and does not impart any undesired toxicological effects [see, e.g. Berge, S. M., et al. (1977) J. Pharm. Sci. 66:1-19]. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical combination of the invention or part thereof also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and non-aqueous carriers that may be employed in the pharmaceutical combinations of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These combinations or parts thereof may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of 100 percent, this amount will range from about 0.01 percent to about 99 percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g. a synergistic combination, a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the anti-LY75 antibody, the dosage ranges from about 0.5 to 5 mg/kg, for example, 1 to 3 mg/kg most preferably 3 mg/kg of the host body weight. For example dosages can be 1 mg/kg body weight, 2 mg/kg body weight, 3 mg/kg body weight, 4 mg/kg body weight, 5 mg/kg body weight. An exemplary treatment regime entails administration once per week, once every two weeks, once every three weeks, once every four weeks, once a month. Preferred dosage regimens for an anti-LY75 antibody of the invention include 3 mg/kg body weight via intravenous administration, with the antibody being given every three to 4 weeks. In one preferred embodiment the anti-LY75 antibody of the invention is administered at 3 mg/kg body weight every 21 days.

In some embodiments, the platin (or a pharmaceutically acceptable salt thereof) is administered at a dosage of 50 to 250 mg/m². The person skilled in the art will be able to calculate the correct dosage regime for the platin drug, e.g. cisplatin, carboplatin or oxaliplatin, for the individual patient based upon their size and body weight and the cancer to be treated.

Preferably, the combination of the invention is a synergistic combination. The skilled person will understand that a synergistic combination is one wherein the effect of the combination is greater than the sum of the effects of the individual components.

In particular, there is provided a method of treating cancer in a patient comprising simultaneously, sequentially or separately administering to a patient in need thereof therapeutically effective synergistic amounts of the components of the pharmaceutical combination of the invention.

Also provided is a pharmaceutical combination of the invention for use in the treatment of cancer, wherein synergistic amounts of the components of the pharmaceutical combination of the invention are simultaneously, separately or sequentially administered to the patient for the treatment of the cancer.

Also provided is the use of synergistic amounts of the components of the pharmaceutical combination of the invention in the manufacture of a pharmaceutical combination for simultaneous, separate or sequential use for the treatment of cancer. Also provided is a synergistic pharmaceutical combination of the invention for use in therapy or for use as a medicament.

In some methods, two or more anti-LY75 monoclonal antibodies with different binding specificities are administered simultaneously, in which case the dosage of each antibody administered falls within the ranges indicated. Antibody is usually administered on multiple occasions. Intervals between single dosages can be, for example, weekly, once every 2 weeks, once every 3 weeks, monthly. Intervals can also be irregular as indicated by measuring blood levels of antibody to the target antigen in the patient. In some methods, dosage is adjusted to achieve a plasma antibody concentration of about 1-1000 μg/ml, 5-750 μg/ml, 10-600 μg/ml, 15-500 μg/ml, 20-400 μg/ml and in some methods about 25-300 μg/ml.

Alternatively, the anti-LY75 antibodies can be administered as sustained release formulations, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical combinations of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-LY75 antibody preferably results in a decrease in severity of disease symptoms, an increase in frequency and duration of disease symptom-free periods, or a prevention of impairment or disability due to the disease affliction. For example, for the treatment of the LY75 mediated tumors, a “therapeutically effective dosage” preferably inhibits cell growth or tumor growth by at least about 20%, at least about 30%, more preferably by at least about 40%, at least about 50% even more preferably by at least about 60%, at least about 70% and still more preferably by at least about 80% or at least about 90%, relative to untreated subjects. The ability of a compound to inhibit tumor growth can be evaluated in an animal model system predictive of efficacy in human tumors. Alternatively, this property of a composition can be evaluated by examining the ability of the compound to inhibit cell growth, such inhibition can be measured in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

A pharmaceutical combination of the present invention can be administered via one or more routes of administration using one or more of a variety of methods known in the art. The components of the pharmaceutical combination of the invention may be administered by the same route or by different routes. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, the anti-LY75 antibody can be administered via a non-parenteral route, such as a topical, epidermal or mucosal route of administration, for example, intranasally, orally, vaginally, rectally, sublingually or topically.

Preferably, the platin or a pharmaceutically acceptable salt thereof is administered intravenously.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art [see, e.g. Sustained and Controlled Release Drug Delivery Systems (1978) J. R. Robinson, ed., Marcel Dekker, Inc., N.Y].

Therapeutic compositions can be administered with medical devices known in the art. For example, in a preferred embodiment, the components of the pharmaceutical composition of the present invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-LY75 antibodies can be formulated to ensure proper distribution in vivo. For example, the blood-brain barrier (BBB) excludes many highly hydrophilic compounds. To ensure that the therapeutic compounds cross the BBB (if desired), they can be formulated, for example, in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs, thus enhance targeted drug delivery [see, e.g. V. V. Ranade (1989) J. Clin. Pharmacol. 29:685]. Exemplary targeting moieties include folate or biotin (see, e.g. U.S. Pat. No. 5,416,016.); mannosides [Umezawa et al. (1988) Biochem. Biophys. Res. Commun. 153:1038]; antibodies [P. G. Bloeman et al. (1995) FEBS Lett. 357:140; M. Owais et al. (1995) Antimicrob. Agents Chemother. 39:180]; surfactant protein A receptor [Briscoe et al. (1995) Am. J. Physiol. 1233:134]; p120 [Schreier et al. (1994) J. Biol. Chem. 269:9090]; see also K. Keinanen; M. L. Laukkanen (1994) FEBS Lett. 346:123; J. J. Killion; I. J. Fidler (1994) Immunomethods 4:273.

### Uses and Methods

As used herein, the term “subject” is intended to include human and non-human animals. Non-human animals include all vertebrates, e.g. mammals and non-mammals, such as non-human primates, sheep, dogs, cats, cows, horses, chickens, amphibians, and reptiles. Preferred subjects include human patients having disorders mediated by LY75 activity.

The methods are particularly suitable for treating human patients having a disorder associated with the aberrant LY75 expression. Given the expression of LY75 on tumor cells, the combinations and methods of the present invention can be used to treat a subject with a tumorigenic disorder, e.g. a disorder characterized by the presence of tumor cells expressing LY75 or in the manufacture of a medicament for the treatment of such a disorder including, for example pancreatic cancer, ovarian cancer, breast cancer, colorectal cancer, esophageal cancer, skin cancer, thyroid cancer, lung cancer (NSCLC and/or SCLC), kidney cancer, liver cancer, head and neck cancer, bladder cancer, gastric cancer, myeloma, preferably multiple myeloma, leukaemia, including chronic lymphocytic leukaemia and acute myeloid leukaemia, non-Hodgkin's lymphoma, including DLBCL, B-Cell Lymphoma, Follicular Lymphoma, Mantle Cell Lymphoma, Lymphoma of Mucosa-Associated Lymphoid Tissue (MALT), T-Cell/Histiocyte-Rich B-Cell Lymphoma, Burkitt's Lymphoma, Lymphoplasmacytic Lymphoma, Small Lymphocytic Lymphoma, Marginal Zone Lymphoma, T Cell Lymphoma, Peripheral T-Cell Lymphoma, Anaplastic Large Cell Lymphoma and AngioImmunoblastic T-Cell Lymphoma. LY75 has been demonstrated to be internalised on antibody binding as illustrated in Examples 5 and 7 below, thus enabling the anti-LY75 antibodies to be used in any payload mechanism of action e.g. an ADC approach, radioimmunoconjugate, or ADEPT approach.

The anti-LY75 antibodies, generally administered as ADCs, can be used to inhibit or block LY75 function which, in turn, can be linked to the prevention or amelioration of certain disease symptoms, thereby implicating the LY75 as a mediator of the disease. This can be achieved by contacting a sample and a control sample with the anti-LY75 antibody under conditions that allow for the formation of a complex between the antibody and LY75. Any complexes formed between the antibody and the LY75 are detected and compared in the sample and the control.

Suitable routes of administering the antibody compositions (e.g. monoclonal antibodies, and immunoconjugates) in vivo and in vitro are well known in the art and can be selected by those of ordinary skill. For example, the antibody compositions can be administered by injection (e.g. intravenous or subcutaneous). Suitable dosages of the molecules used will depend on the age and weight of the subject and the concentration and/or formulation of the antibody composition.

The pharmaceutical combinations of the invention can also be administered together with serum and/or complement. These compositions can be advantageous when the complement is located in close proximity to the antibodies. Alternatively, the antibodies, and the complement or serum can be administered separately.

Also within the scope of the present invention are kits comprising the components of the pharmaceutical combination of the invention, together with instructions for use. The kit can further contain one or more additional reagents, such as an immunosuppressive reagent, a cytotoxic agent or a radiotoxic agent, or one or more additional antibodies (e.g. an antibody having a complementary activity which binds to an epitope in the LY75 antigen distinct from the first antibody).

Accordingly, patients treated with pharmaceutical combinations of the invention can be additionally administered (prior to, simultaneously with, or following administration of an antibody disclosed herein) with another therapeutic agent, such as a cytotoxic or radiotoxic agent, which enhances or augments the therapeutic effect of the antibodies.

In other embodiments, the subject can be additionally treated with an agent that modulates, e.g. enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

All references cited in this specification, including without limitation all papers, publications, patents, patent applications, presentations, texts, reports, manuscripts, brochures, books, internet postings, journal articles, periodicals, product fact sheets, and the like, one hereby incorporated by reference into this specification in their entireties. The discussion of the references herein is intended to merely summarize the assertions made by their authors and no admission is made that any reference constitutes prior art and Applicants' reserve the right to challenge the accuracy and pertinence of the cited references.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the dependent claims.

The present invention is further illustrated by the following examples which should not be construed as further limiting.

### Example 1: Structural Characterization of Monoclonal Antibodies to LY75

The cDNA sequences encoding the heavy and light chain variable regions of the LY75_A1 monoclonal antibody were obtained using standard PCR techniques and were sequenced using standard DNA sequencing techniques.

The antibody sequences may be mutagenized to revert back to germline residues at one or more residues.

The nucleotide and amino acid sequences of the heavy chain variable region of LY75_A1 are shown in SEQ ID NO: 3 and 1, respectively. The nucleotide and amino acid sequences of the light chain variable region of LY75_A1 are shown in SEQ ID NO: 4 and 2, respectively.

The amino acid and nucleotide sequences of the heavy chain of LY75_A1 are shown in SEQ ID NOs: 24 and 26, respectively. The amino acid and nucleotide sequences of the light chain of LY75_A1 are shown in SEQ ID NOs: 25 and 27, respectively.

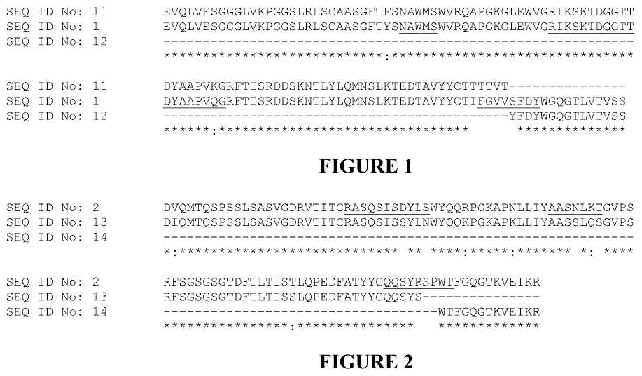

Comparison of the LY75_A1 heavy chain immunoglobulin sequence to the known human germline immunoglobulin heavy chain sequences demonstrated that the LY75_A1 heavy chain utilizes a V_{H} segment from human germline V_{H} 3-15 and a JH segment from human germline JH JH4. Further analysis of the LY75_A1 V_{H} sequence using the Kabat system of CDR region determination led to the delineation of the heavy chain CDR1, CDR2 and CDR3 regions as shown in SEQ ID NOs: 5, 6 and 7, respectively. The alignments of the LY75_A1 CDR1, CDR2 and CDR3 V_{H} sequences to the germline V_{H} 3-15 and germline JH JH4 sequence are shown in FIG. 1.

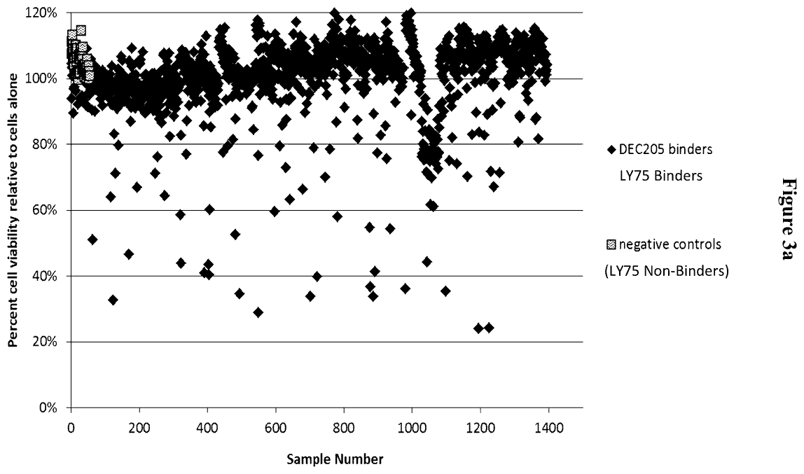

Comparison of the LY75_A1 light chain immunoglobulin sequence to the known human germline immunoglobulin light chain sequences demonstrated that the LY75_A1 light chain utilizes a V_{K} segment from human germline V_{K} O12 and a J_{K} segment from human germline J_{K} J_{K}4. Further analysis of the LY75_A1 V_{K} sequence using the Kabat system of CDR region determination led to the delineation of the light chain CDR1, CDR2 and CDR3 regions as shown in SEQ ID NOs: 8, 9 and 10, respectively. The alignments of the LY75_A1 CDR1, CDR2 and CDR3 V_{K} sequences to the germline V_{K} O12 and germline J_{K} J_{K}4 sequences are shown in FIG. 2.

### Example 2: Immunohistochemistry Using Monoclonal Antibody to LY75

Using the human monoclonal antibodies specific to LY75, immunohistochemistry was performed on FFPE HT-29 and A549 cell pellets, FFPE non-Hodgkin's lymphoma and pancreatic cancer arrays, and fresh frozen lymphoma/leukaemia tumours, ovarian cancer, pancreatic cancer, and breast cancer sections and a normal tissue array.

### Materials and Methods

### Materials

Xylenes (X5P-1 gal) from Fisher Scientific, PA, USA.
Histoprep 100% ethanol (HC-800-1GAL) from Fisher Scientific, PA, USA.
10×Citrate buffer for heat induced epitope retrieval (AP9003125) from Thermo Scientific, MA, USA.
Thermo Scientific* Pierce* Peroxidase Suppressor (35000) from Thermo Scientific, MA, USA.
Serum free protein block (X0909) from Dako, CA, USA
Secondary antibody: goat anti-human IgG Fab-FITC conjugated (109-097-003) from Jackson Immunoresearch, PA, USA
Chrome pure Human IgG, whole molecule (09-000-003) from Jackson Immunoresearch, PA, USA
Tertiary antibody: mouse anti-FITC (ab10257) from Abcam, MA, USA
Purified human IgG isotype control (1-001A) from R&D Systems, MN, USA
Tween-20 (BP337-100) from Fisher Scientific, PA, USA
Acetone (BP2403-4) from Fisher Scientific, PA, USA
Dual Link EnVision+ HRP-conjugated polymer, Mouse and Rabbit (K4063) from Dako, CA, USA.
DAB 2-solution kit (882014) from Invitrogen, NY, USA.
Harris Hematoxylin (23-245-677) from Fisher Scientific, PA, USA.
Faramount mounting media (S302580) from Dako, CA, USA.
   Tissue sections and arrays were purchased from US Biomax Inc., MD, USA or Origene, MD, USA.

### Preparation of FFPE Slides: Deparaffinisation and Rehydration

FFPE slides were deparaffinised in xylene (2×3 minutes) then rehydrated through 1:1 xylene: 100% ethanol (1×3 minutes), 100% ethanol (2×3 minutes), 95% ethanol (1×3 minutes), 70% ethanol (1×3 minutes), 50% ethanol (1×3 minutes), and tap water (1×3 minutes).

### Preparation of FFPE Slides: Antigen Retrieval (Microwave).

The LY75 antigen was retrieved using microwave heat, high power until boiling then low power for 10 minutes in 50 mL 1×citrate buffer in a Coplin jar. Slides were then left to cool to room temperature for a further 15 min, then washed in tap water, 3 minutes. Circles were drawn around each tissue section/TMA with a hydrophobic barrier pen and slides were then washed 3 times in PBS, 3 minutes each wash.

### Preparation of FF Slides

Slides were removed from storage at −80 C and allowed to dry at room temperature in the fume hood for 20-30 minutes. The slides were fixed for 10 min in ice cold acetone at −20 C, then allowed to dry for 20 min in the fume hood at room temperature. Slides were washed and rehydrated in PBS, 3 washes for 3 min each. Sections were outlined with a hydrophobic barrier pen.

### Preparation of Antibody Complexes

The primary anti-LY75 antibody was diluted in serum free protein block (SFPB) to obtain a solution with a concentration 20-fold greater than the final desired concentration (20 μg/mL for 1 μg/mL final). The secondary antibody, goat anti-human immunoglobulin G (IgG) antigen-binding fragment (Fab), was prepared similarly in SFPB to create a solution of equal concentration.

Equal volumes of primary and secondary antibodies were combined in a labelled tube, gently mixed, and incubated for 3 minutes at room temperature, resulting in a primary antibody concentration 10-fold greater than the desired final concentration (10 μg/mL for 1 μg/mL final). This mixture was diluted 1:5 with SFPB, gently mixed, and incubated for 30 minutes at room temperature, resulting in a primary antibody concentration twice that of the desired final concentration (2 μg/mL for 1 μg/mL final).

To produce the final staining complexes, a 1% (10 μg/μL) solution of human IgG was prepared in SFPB and equal volume added to the primary/secondary antibody mixture. This combination was gently mixed and incubated at room temperature for 30 minutes, diluting by half the primary antibody concentration of the primary/secondary antibody mixture and resulting in the desired final primary antibody concentration (1 μg/mL).

### Immunostaining

Meanwhile, endogenous tissue peroxidase activity was blocked by incubating tissues with peroxidase suppressor for 5-10 minutes at RT in a humidified chamber. Slides were then washed in PBS 3×3 minutes each wash. Tissues were incubated in SFPB for 30 minutes at room temperature in a humidified chamber. Final staining complexes were applied to each tissue section and/or microarray, and the slides were incubated for 30 min at room temperature in a humidified chamber. Slides were then washed once in PBS and once in PBST (PBS+0.125% Tween-20), 3 minutes each wash. The tertiary antibody mouse anti-FITC, was applied at 2 μg/mL concentration for 30 min, room temperature, in a humidified chamber. Sections were then washed once in PBS and once in PBST, 3 min each wash. Dual Link EnVision+ anti-mouse/rabbit-HRP-conjugated polymer was then applied to the tissues and the slides were incubated for 30 min at room temperature in a humidified chamber. Slides were then washed once in PBS, once in PBST, 3 minutes each wash. Tissues were incubated in DAB solution prepared according to the manufacturer's instructions at room temperature for 10 min. Slides were then washed once in running tap water for 2 minutes and once in PBS for 3 minutes. The slides were counterstained with Hematoxylin for 30 seconds at room temperature, and washed with running tap water. The slides were dried at room temperature for 30 minutes and coverslips were then mounted onto the slides using Faramount mounting media.

### Results

LY75_A1 showed positivity in FFPE Triple Negative breast cancer samples, where 77% of the sections showed positive staining and 55% exhibited robust (+++) staining.

Staining for LY75 in FF normal tissues was generally absent to low. Ductal epithelium of the breast, salivary gland, and pancreas exhibited marked low to moderate staining, and the spleen stained low positive. Therefore antibodies directed to LY75 may have utility as therapeutics and diagnostics in some of the tested cancers and possibly other cancer types showing expression of LY75.

### Example 3: Efficacy of DM1-Conjugated Anti-LY75 Monoclonal Antibodies in HT-29 Cells

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/mL.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

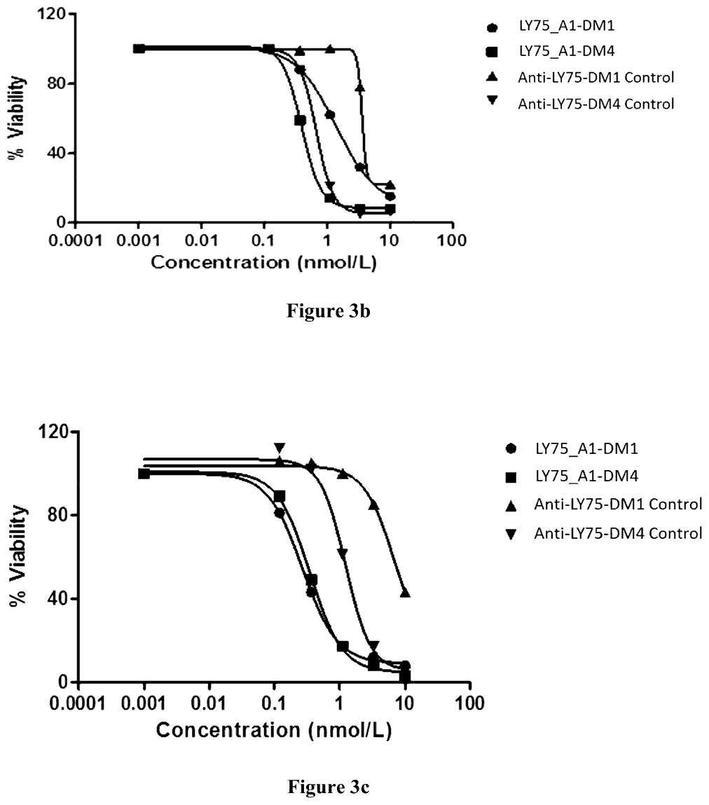

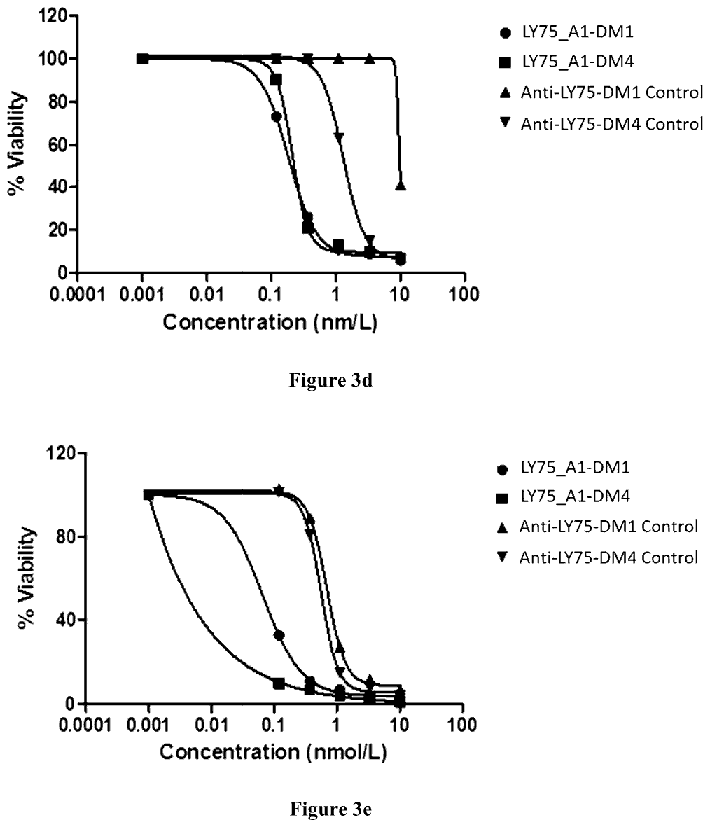

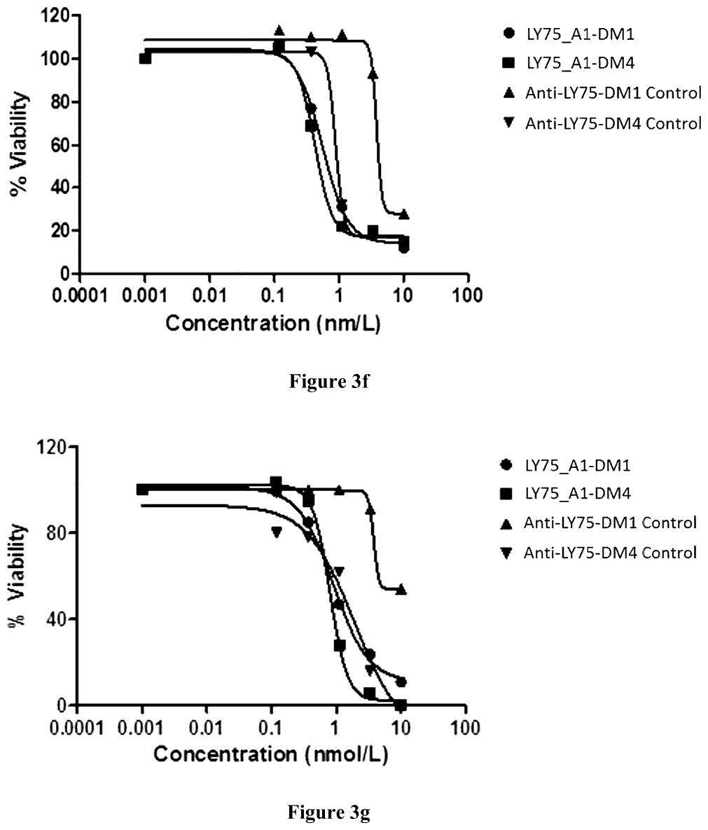

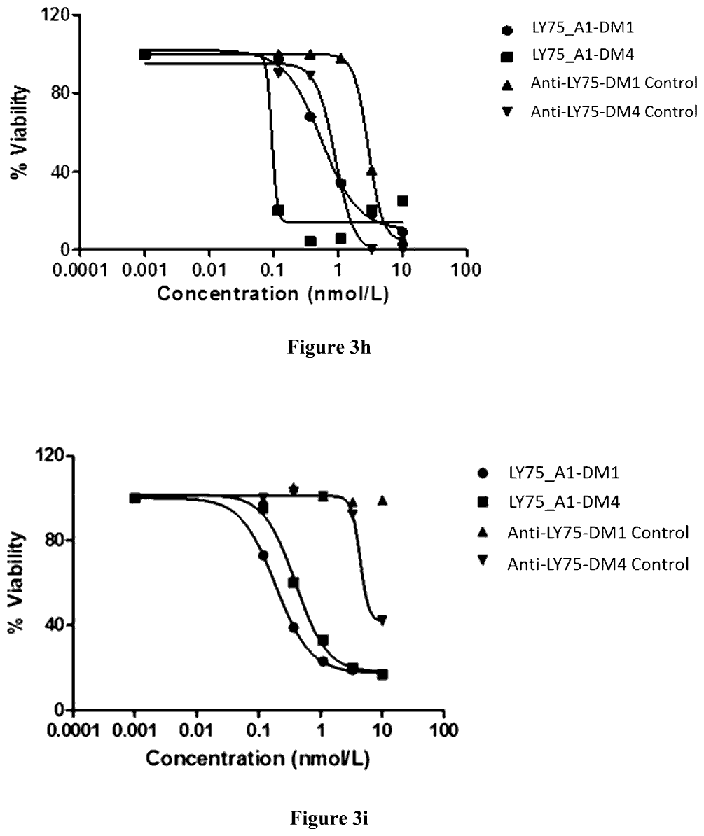

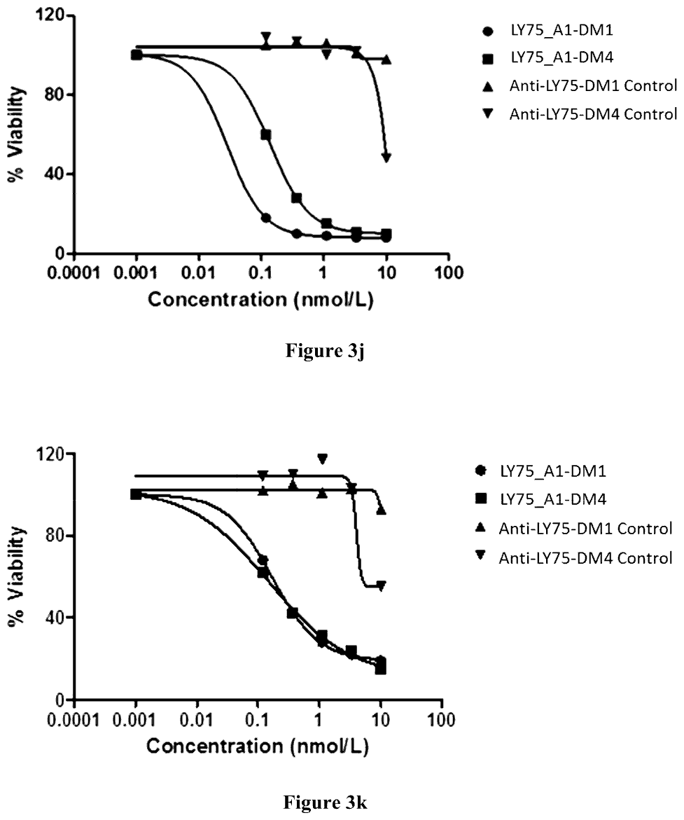

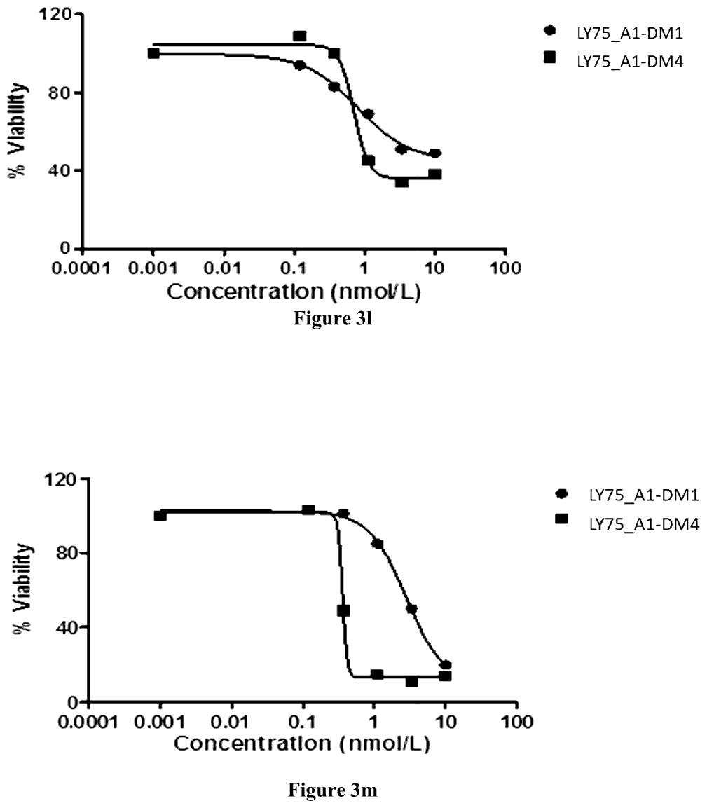

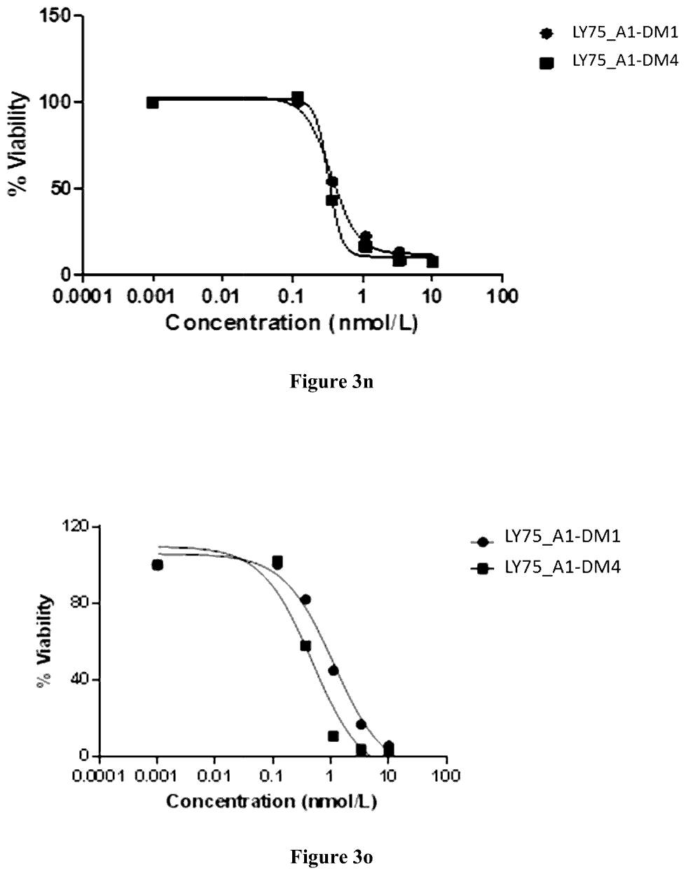

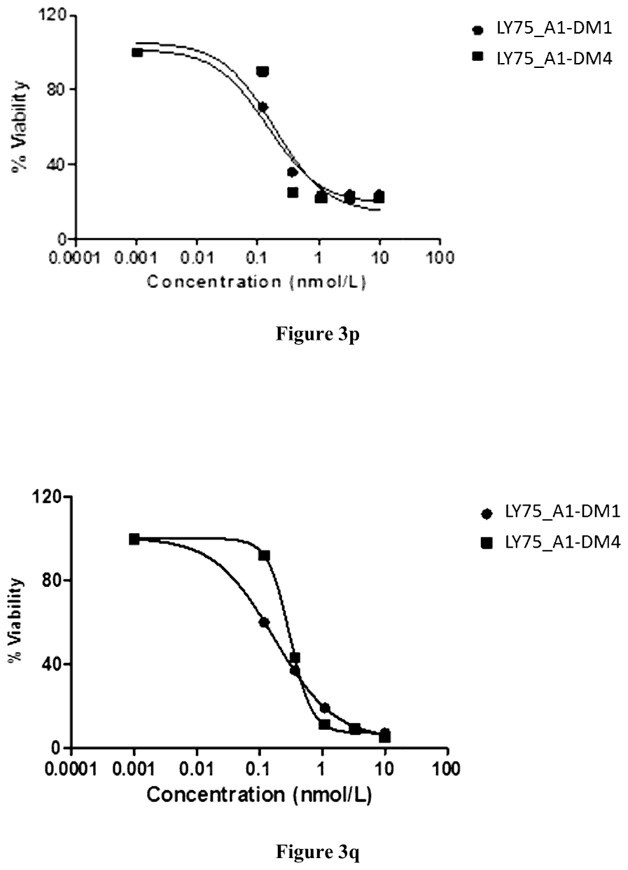

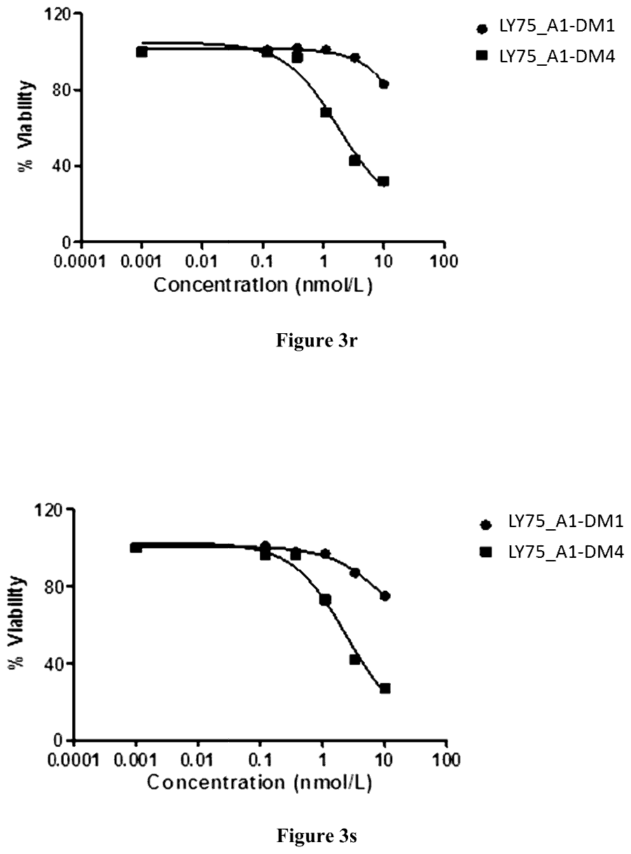

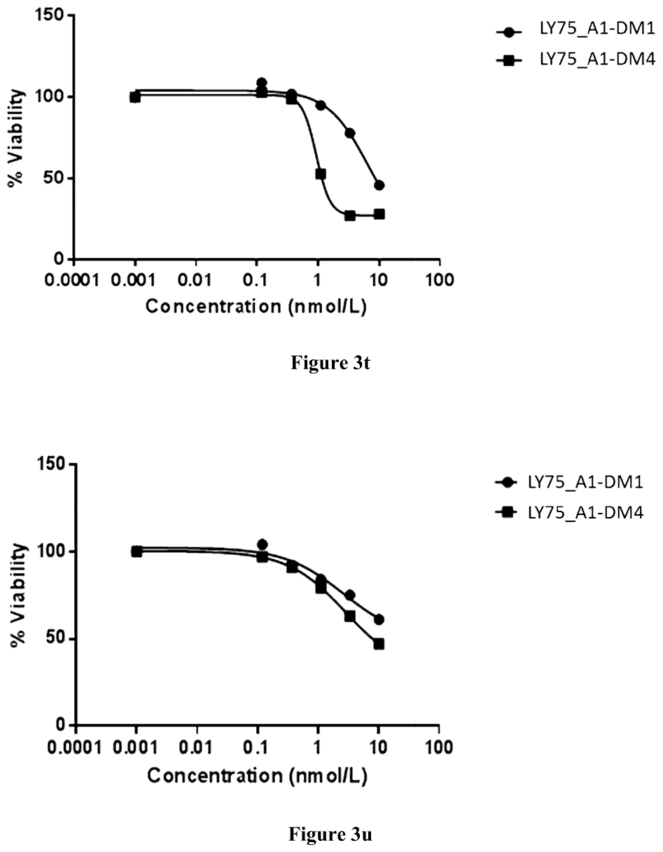

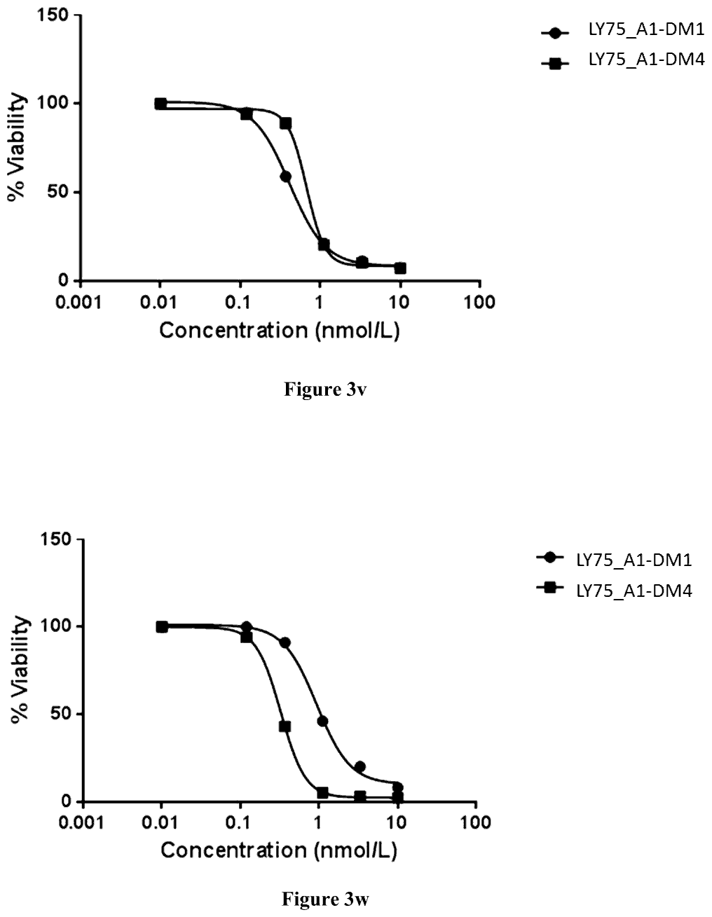

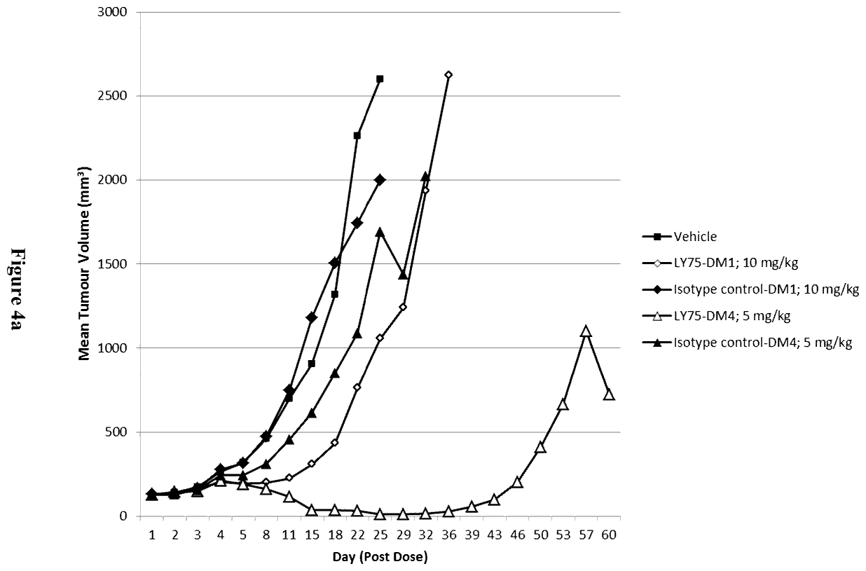

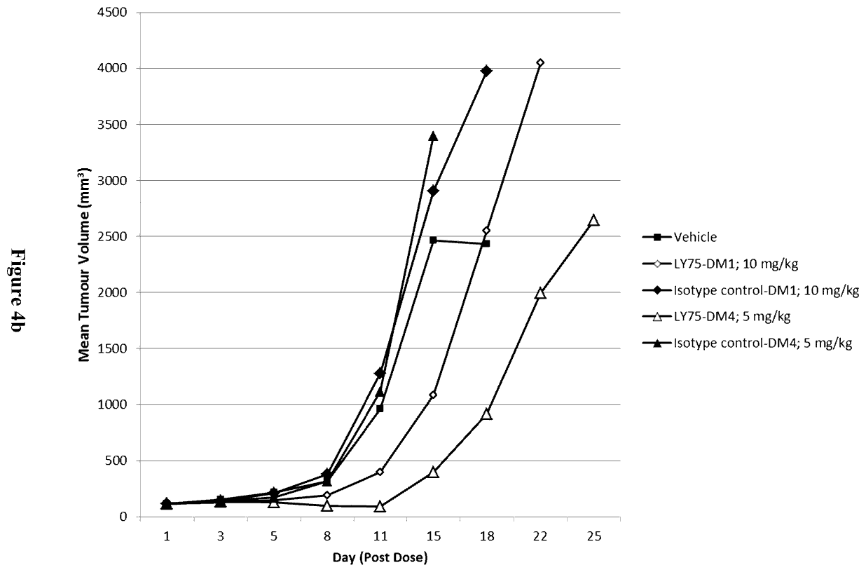

The results depicted in FIG. 3a show a subpopulation of antibodies, know to bind to LY75, which can induce cell kill of HT-29 cells. This suggests while antibodies can bind to LY75 only a few display efficacy when conjugated to DM1. Antibodies where then chosen from the subpopulation for further cytotoxic activity analysis.

### Example 4: Efficacy of DM1-Conjugated and DM4-Conjugated Anti-LY75 Monoclonal Antibodies in Colorectal Cancer Cells

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/m.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3b shows the cytotoxic activity of anti-LY75 antibodies conjugated to DM1 and DM4 towards HT-29 cells These results demonstrate an increase in cytotoxic activity proportional to antibody concentration and other anti-LY75 antibodies conjugated to a toxin (selected from Example 1).

### Example 5: Efficacy of DM1-Conjugated and DM4-Conjugated Anti-LY75 Monoclonal Antibodies in Lymphoma Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/m. 50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3c shows the cytotoxic activity of anti-LY75 antibodies conjugated to DM1 and DM4 towards RAJI cells. FIG. 3d shows the cytotoxic activity of anti-LY75 antibodies conjugated to DM1 and DM4 towards Namalwa cells. FIG. 3e shows the cytotoxic activity of anti-LY75 antibodies conjugated to DM1 and DM4 towards Karpas 299 cells. These results demonstrate an increase in cytotoxic activity proportional to antibody concentration and other anti-LY75 antibodies conjugated to DM1 and DM4 (selected from Example 1).

### Example 6: Efficacy of DM1-Conjugated and DM4-Conjugated Anti-LY75 Monoclonal Antibodies in Pancreatic Cancer Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/m.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3f shows the cytotoxic activity of anti-LY75 antibodies conjugated to DM1 and DM4 towards BxPC3 cells. FIG. 3g shows the cytotoxic activity of anti-LY75 antibodies conjugated to DM1 and DM4 towards HupT4 cells. FIG. 3h shows the cytotoxic activity of anti-LY75 antibodies conjugated to DM1 and DM4 towards HPAFFII cells. These results demonstrate an increase in cytotoxic activity proportional to antibody concentration and other anti-LY75 antibodies conjugated to DM1 and DM4 (selected from Example 1).

### Example 7: Efficacy of DM1-Conjugated and DM4-Conjugated Anti-LY75 Monoclonal Antibodies in Chronic Lymphocytic Leukaemia Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/m.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3i shows the cytotoxic activity of anti-LY75 antibodies conjugated to DM1 and DM4 towards EHEB cells. FIG. 3j shows the cytotoxic activity of anti-LY75 antibodies conjugated to DM1 and DM4 towards Mec-1 cells. These results demonstrate an increase in cytotoxic activity proportional to antibody concentration and other anti-LY75 antibodies conjugated to DM1 and DM4 (selected from Example 1).

### Example 8: Efficacy of DM1-Conjugated and DM4-Conjugated Anti-LY75 Monoclonal Antibodies in Acute Monocytic Leukaemia Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/m.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3k shows the cytotoxic activity of anti-LY75 antibodies conjugated to DM1 and DM4 towards AML-193 cells. These results demonstrate an increase in cytotoxic activity proportional to antibody concentration and other anti-LY75 antibodies conjugated to DM1 and DM4 (selected from Example 1).

### Example 9: Efficacy of DM1-Conjugated and DM4-Conjugated Anti-LY75 Monoclonal Antibodies in Breast Cancer Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/m.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3l shows the cytotoxic activity of anti-LY75 antibodies conjugated to DM1 and DM4 towards HCC 70 (ER negative, PR negative and Her2 negative) cells. FIG. 3m shows the cytotoxic activity of anti-LY75 antibodies conjugated to DM1 and DM4 towards HCC 1806 (ER negative, PR negative and Her2 negative) cells. FIG. 3n shows the cytotoxic activity of anti-LY75 antibodies conjugated to DM1 and DM4 towards MDA-MB-468 cells. These results demonstrate an increase in cytotoxic activity proportional to antibody concentration.

### Example 10: Efficacy of DM1-Conjugated and DM4-Conjugated Anti-LY75 Monoclonal Antibodies in Bladder Cancer Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/mL.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 30 shows the cytotoxic activity of anti-LY75 antibodies conjugated to DM1 and DM4 towards RT4 cells. FIG. 3p shows the cytotoxic activity of anti-LY75 antibodies conjugated to DM1 and DM4 towards 5637 cells. FIG. 3q shows the cytotoxic activity of anti-LY75 antibodies conjugated to DM1 and DM4 towards SW780 cells. These results demonstrate an increase in cytotoxic activity proportional to antibody concentration.

### Example 11: Efficacy of DM1-Conjugated and DM4-Conjugated Anti-LY75 Monoclonal Antibodies in Head and Neck Cancer Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/mL.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3r shows the cytotoxic activity of anti-LY75 antibodies conjugated to DM1 and DM4 towards SCC-9 cells. These results demonstrate an increase in cytotoxic activity proportional to antibody concentration.

### Example 12: Efficacy of DM1-Conjugated and DM4-Conjugated Anti-LY75 Monoclonal Antibodies in Oesophageal Cancer Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/mL.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3s shows the cytotoxic activity of anti-LY75 antibodies conjugated to DM1 and DM4 towards OE 19 cells. These results demonstrate an increase in cytotoxic activity proportional to antibody concentration.

### Example 13: Efficacy of DM1-Conjugated and DM4-Conjugated Anti-LY75 Monoclonal Antibodies in Ovarian Cancer Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/mL.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3t shows the cytotoxic activity of anti-LY75 antibodies conjugated to DM1 and DM4 towards OVCAR-3 cells. FIG. 3u shows the cytotoxic activity of anti-LY75 antibodies conjugated to DM1 and DM4 towards SK-OV-3 cells. These results demonstrate an increase in cytotoxic activity proportional to antibody concentration.

### Example 14: Efficacy of DM1-Conjugated and DM4-Conjugated Anti-LY75 Monoclonal Antibodies in Multiple Myeloma Cell Lines

### Materials

Cell stripper (Non-enzymatic cell dissociation) (MT-25-056CI) from Fisher Scientific, PA, USA.
PBS pH 7.4 (1×) (SH30028LS) from Fisher Scientific, PA, USA.
RPMI 1640 Media (MT-10⁻⁰⁴¹-CM) from Fisher Scientific, PA, USA.
Cell Titer Glo (G7572) from Promega, WI, USA.

### Method

Cells were dissociated using cell stripper and counted. 5e3 cells/well were spun down into a pellet (for suspension cells, more can be used depending on the doubling time of the cells, such as 10e3 cells/well). The pellet was resuspended in culture media to a concentration of 1e5 cells/mL.

50 ul/well cell suspension was added to wells of a 96-well white sided, clear bottomed plate. Antibodies were diluted and titrated to 8 points (3-fold titrations) corresponding to concentrations between 0-20 nM (twice the test concentrations). Diluted antibodies or media (for untreated samples) (50 ul/well) were added to the appropriate wells. Excess media (200 ul/well) was added to the outside rows and columns of the plate to prevent evaporation. The plate was incubated for 72 h at 37 C.

The plate was removed from the incubator and incubated at room temperature for 30 minutes. Meanwhile Cell Titer Glo solution was thawed. The plate was flicked and washed 1× with 100 ul/well PBS (for suspension cells, plate is centrifuged first to pellet cells). 100 ul/well PBS and 100 ul Cell titer glo was added to each well and triturated to mix. The plate was incubated in the dark at room temperature for 15 minutes and visualized by microscopy to ensure efficient cell lysis occurred. The plate was then read on a Glomax luminometer.

### Results

FIG. 3v shows the cytotoxic activity of anti-LY75 antibodies conjugated to DM1 and DM4 towards MOLP-8 cells. FIG. 3w shows the cytotoxic activity of anti-LY75 antibodies conjugated to DM1 and DM4 towards RPMI8226 cells. These results demonstrate an increase in cytotoxic activity proportional to antibody concentration.

### Example 15: Efficacy of DM1-Conjugated and DM4-Conjugated Anti-LY75 Monoclonal Antibodies in Raji Xenograft Models

The efficacy of LY75_DM1 and LY75_DM4 were tested in subcutaneous Raji Burkitt's lymphoma SCID mouse xenograft model.

Immunodeficient SCID mice were inoculated subcutaneously with Raji (human Burkitt's lymphoma) tumour cells. Tumours were allowed to establish and mice were sorted into five treatment groups of 3-6 mice per group. When the mean tumour volume reached an average size of 129-132 mm3 per group, each group was treated with one of the following compounds, administered intravenously at the indicated dosages: Group 1 (Vehicle; phosphate buffered saline (PBS)); Group 2 (LY75_DM1; 10 mg/kg), Group 3 (Isotype control-DM1; 10 mg/kg), Group 4 (LY75_DM4; 5 mg/kg), Group 5 (isotype control-SPBDDM4; 5 mg/kg). A second dose was administered one week later. Body weights (BW) were monitored, the mice were examined frequently for health and adverse side effects, and tumours were measured twice weekly. Mice were euthanized when their tumours reached the tumour volume endpoint of 2000 mm3 or after 60 days, whichever came first. Efficacy was determined from tumour growth delay (TGD), the increase in median time-to-endpoint (TTE) and from logrank analysis of differences in Kaplan Meier survival curves in ADC-treated versus PBS-treat mice. The first five vehicle-treated control mice to reach endpoint were sampled for tumours that were processed by formalin fixation and paraffin embedded.

### Results

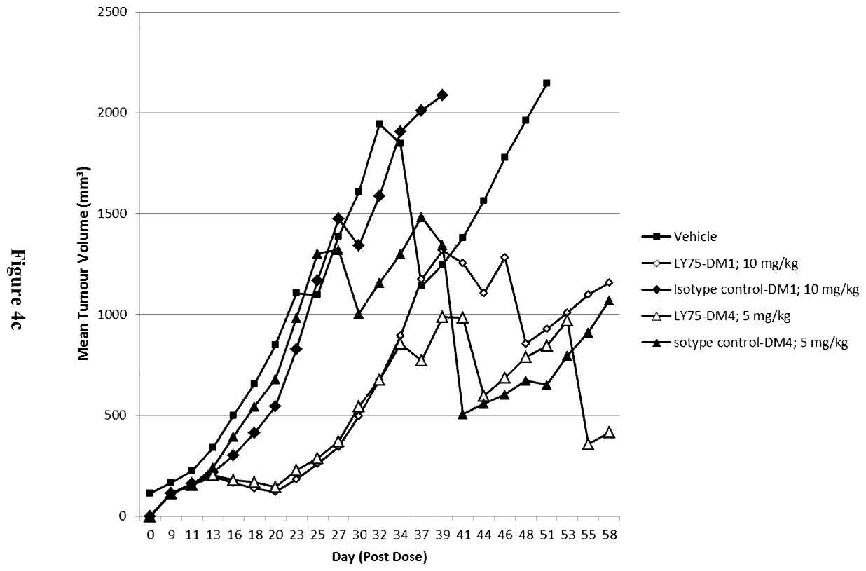

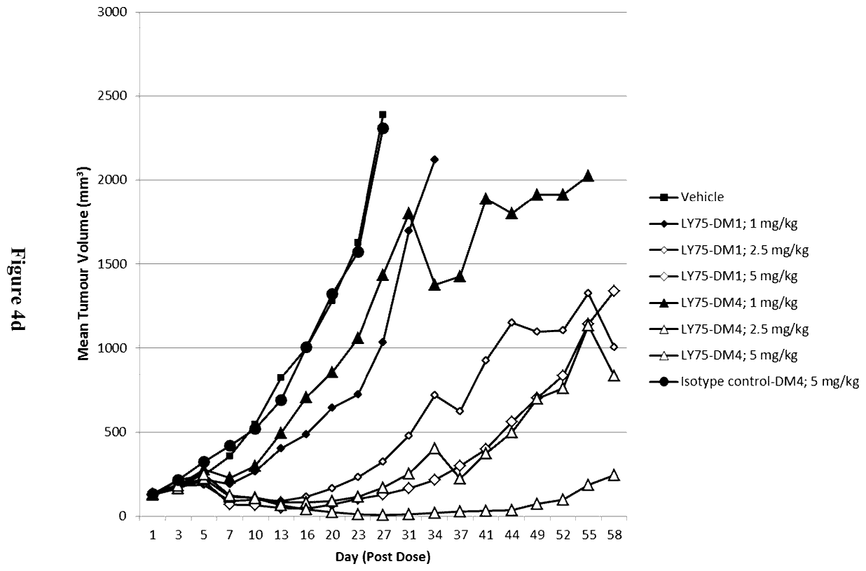

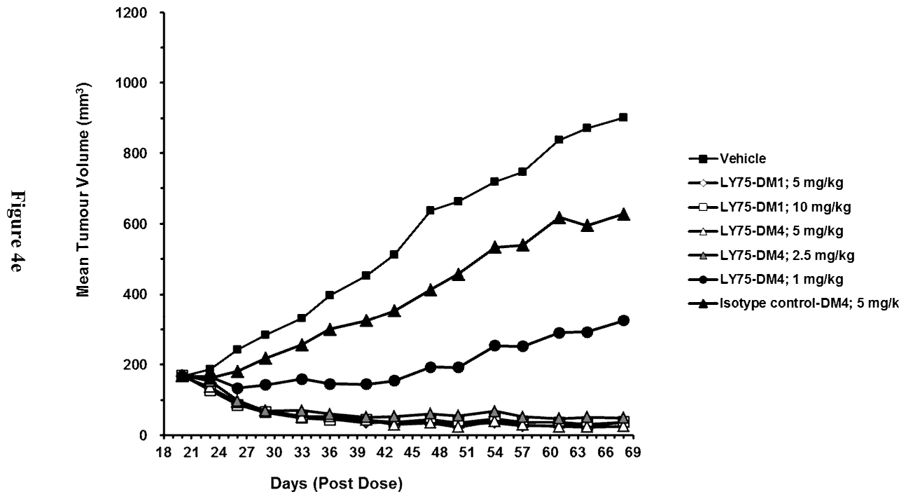

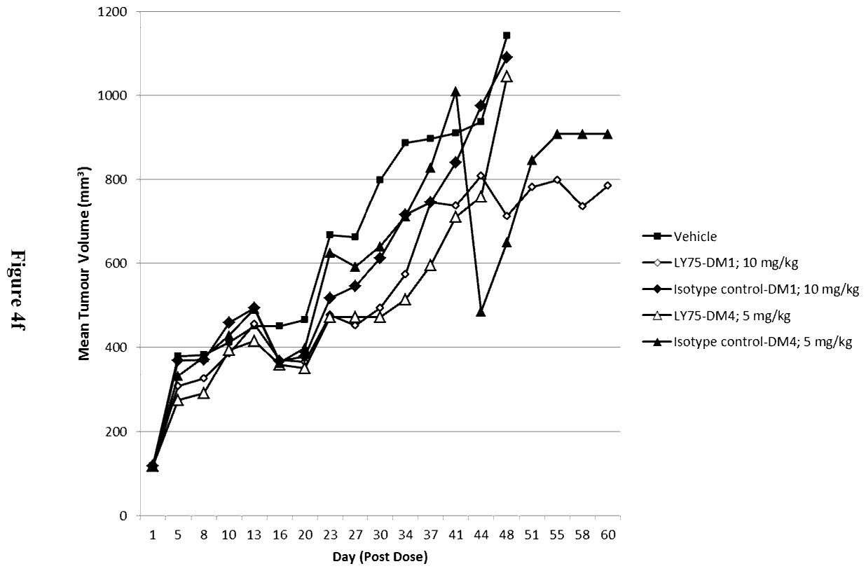

FIG. 4a shows LY75_DM1 and LY75_DM4 each demonstrated significant anti-tumour activity and significantly extended survival in the Raji Burkitt's lymphoma SCID mouse xenograft model compared to controls; however, the 5 mg/kg LY75_DM4 doses were significantly more effective than the 10 mg/kg doses of LY75_DM1, resulting in 5 of 6 mice with complete but transient tumour regression. All treatments were well-tolerated and no clinical signs of toxicity were observed. These data suggest the potential for ADCs directed towards LY75, for example LY75_DM1 and LY75_DM4, to provide clinical benefit in the treatment of human non-Hodgkin lymphoma cancer patients.

### Example 16: Efficacy of DM1-Conjugated and DM4-Conjugated Anti-LY75 Monoclonal Antibodies in Namalwa Xenograft Models

The efficacy of LY75_DM1 and LY75_DM4 were tested in subcutaneous Namalwa Burkitt's lymphoma SCID mouse xenograft model.

Immunodeficient SCID mice were inoculated subcutaneously with Namalwa (human Burkitt's lymphoma) tumour cells. Tumours were allowed to establish and mice were sorted into five treatment groups of 6 mice per group. When the mean tumour volume reached an average size of 114 mm3 per group, each group was treated with one of the following compounds, administered intravenously at the indicated dosages: Group 1 (Vehicle; phosphate buffered saline (PBS)); Group 2 (LY75_DM1; 10 mg/kg), Group 3 (Isotype control-DM1; 10 mg/kg), Group 4 (LY75_DM4; 5 mg/kg), Group 5 (isotype control-SPBDDM4; 5 mg/kg). Body weights (BW) were monitored, the mice were examined frequently for health and adverse side effects, and tumours were measured twice weekly. Mice were euthanized when their tumours reached the tumour volume endpoint of 2000 mm3 or after 60 days, whichever came first. Efficacy was determined from tumour growth delay (TGD), the increase in median time-to-endpoint (TTE), and from log rank analysis of differences in Kaplan Meier survival curves in ADC-treated versus PBS-treated mice. The first five vehicle-treated control mice to reach endpoint were sampled for tumours that were processed by formalin fixation and paraffin embedded.

### Results

FIG. 4b shows LY75_DM1 and LY75_DM4 each demonstrated significant anti-tumour activity and survival extension in the Namalwa Burkitt's lymphoma SCID mouse xenograft model compared to controls; however, the 5 mg/kg LY75_DM4 dose was significantly more effective than the 10 mg/kg dose of LY75_DM1, causing a brief reduction in tumour volume. All treatments were well-tolerated and no clinical signs of toxicity were observed. These data suggest the potential for ADCs directed towards LY75, for example LY75_DM1 and LY75_DM4, to provide clinical benefit in the treatment of human non-Hodgkin lymphoma cancer patients.

### Example 17: Efficacy of DM1-Conjugated and DM4-Conjugated Anti-LY75 Monoclonal Antibodies in Pancreatic Cancer Xenograft Models

The efficacy of LY75_DM1 and LY75_DM4 were tested in the subcutaneous HPAFII pancreatic adenocarcinoma athymic nude mousexenograft model.

Immunodeficient athymic nude mice were inoculated subcutaneously with HPAFII (human pancreatic adenocarcinoma) tumor cells. Tumors were allowed to establish and mice were sorted into five treatment groups of 6 mice per group. When the mean tumor volume reached an average size of ˜114 mm3/group, each group was treated with one of the following compounds, administered intravenously at the indicated dosages: Group 1 (Vehicle; phosphate buffered saline (PBS)); Group 2 (LY75_DM1; 10 mg/kg), Group 3 (Isotype control-DM1; 10 mg/kg), Group 4 (LY75_DM4; 5 mg/kg), Group 5 (isotype control-SPBDDM4; 5 mg/kg). Body weights (BW) were monitored, the mice were examined frequently for health and adverse side effects, and tumors were measured thrice weekly. Mice were euthanized when their tumors reached the tumor volume endpoint of 2000 mm3 or after 90 days, whichever came first. Efficacy was determined from the effect of treatment on tumor volume and from log rank analysis of differences in Kaplan-Meier survival curves in ADC-treated or PBS-treated mice. The tumors were sampled from vehicle-treated control mice and processed by formalin fixation and paraffin embedded.

### Results

FIG. 4c shows LY75_DM1 and LY75_DM4 displayed significant and similarly potent anti-tumor activity and survival extension in the HPAFII nude mouse xenograft model compared to controls. All treatments were well-tolerated and no clinical signs of toxicity were observed. These data suggest the potential for ADCs directed towards LY75, for example LY75_DM1 and LY75_DM4, to provide clinical benefit in the treatment of human pancreatic cancer patients.

### Example 18: Efficacy of DM1-Conjugated and DM4-Conjugated Anti-LY75 Monoclonal Antibodies in Bladder Cancer Xenograft Models

The efficacy of LY75_DM1 and LY75_DM4 were tested in the subcutaneous SW780 human bladder carcinoma SCID mouse xenograft model.

Immunodeficient athymic nude mice were inoculated subcutaneously with HPAFII (human pancreatic adenocarcinoma) tumor cells. Tumors were allowed to establish and mice were sorted into five treatment groups of 6 mice per group. When the mean tumor volume reached an average size of ˜114 mm3/group, each group was treated with one of the following compounds, administered intravenously at the indicated dosages: Group 1 (Vehicle; phosphate buffered saline (PBS)); Group 2 (LY75_DM1; 1 mg/kg), Group 3 (LY75_DM1; 2.5 mg/kg), Group 4 (LY75_DM1; 5 mg/kg), Group 5 (LY75_DM4; 1 mg/kg)), Group 6 (LY75_DM4; 2.5 mg/kg)), Group 7 (LY75_DM4; 5 mg/kg)), Group 8 (isotype control-SPBDDM4; 5 mg/kg). Body weights (BW) were monitored, the mice were examined frequently for health and adverse side effects, and tumors were measured thrice weekly. Mice were euthanized when their tumors reached the tumor volume endpoint of 2000 mm3 or after 90 days, whichever came first. Efficacy was determined from the effect of treatment on tumor volume and from log rank analysis of differences in Kaplan-Meier survival curves in ADC-treated or PBS-treated mice. The tumors were sampled from vehicle-treated control mice and processed by formalin fixation and paraffin embedded.

### Results

FIG. 4d shows LY75_DM1 and LY75_DM4 displayed significant and similarly potent anti-tumor activity and survival extension in the SW780 nude mouse xenograft model compared to controls. All treatments were well-tolerated and no clinical signs of toxicity were observed. These data suggest the potential for ADCs directed towards LY75, for example LY75_DM1 and LY75_DM4, to provide clinical benefit in the treatment of human bladder cancer patients.

### Example 19: Efficacy of DM1-Conjugated and DM4-Conjugated Anti-LY75 Monoclonal Antibodies in Breast Cancer Xenograft Models

The efficacy of LY75_DM1 and LY75_DM4 were tested in the subcutaneous MDA-MB-468 athymic nude mouse xenograft model.

Immunodeficient athymic nude mice were inoculated subcutaneously with MDA-MB-468 (human triple negative breast adenocarcinoma) tumour cells. Tumours were allowed to establish and mice were sorted into seven treatment groups of 10 mice per group. When the mean tumour volume reached an average size of 167 mm3 per group, each group was treated with one of the following compounds, administered intravenously at the indicated dosages: Group 1 (Vehicle; 20 mM sodium succinate, pH 5.0, 6% trehalose, 0.04% polysorbate); Group 2 (LY75_DM1; 5 mg/kg), Group 3 (LY75_DM1; 10 mg/kg), Group 4 (LY75_DM4; 5 mg/kg), Group 5 (LY75_DM4; 2.5 mg/kg), Group 6 (LY75_DM4; 1 mg/kg), Group 7 (Isotype control-DM4; 5 mg/kg). Body weights (BW) were monitored, the mice were examined frequently for health and adverse side effects, and tumours were measured twice weekly. Mice were euthanized 82 days after tumour inoculation. Efficacy was determined from anti-tumour activity (mean tumour size in treatment group/mean tumour size in control group×100) and the increase in mean time-to-endpoint (TTE) in ADC-treated versus PBS-treated mice. The five largest tumours in vehicle-treated control mice on day 71 post inoculation were sampled processed by formalin fixation and paraffin embedded.

### Results

FIG. 4e shows LY75_DM1 and LY75_DM4 each demonstrated dramatic anti-tumour activity in the MDA-MB-468 nude mouse xenograft model compared to controls. Dose dependent activity was observed with LY75_DM4, where 2.5 and 5 mg/kg were much more potent than 1 mg/kg. At 5 mg/kg, LY75_DM1 and LY75_DM4 were similarly effective. Sustained regressions in mean tumour volume were observed for LY75_DM1 at 10 and 5 mg/kg and LY75_DM4 at 5 and 2.5 mg/kg. All treatments were well-tolerated and no clinical signs of toxicity were observed. These data suggest the potential for ADCs directed towards LY75, for example LY75_DM1 and LY75_DM4, to provide clinical benefit in the treatment of human triple negative breast cancer patients.

### Example 20: Efficacy of DM1-Conjugated and DM4-Conjugated Anti-LY75 Monoclonal Antibodies in Colorectal Cancer Xenograft Models

The efficacy of LY75_DM1 and LY75_DM4 were tested in the subcutaneous COLO205 colorectal adenocarcinoma athymic nude mouse xenograft model.

Immunodeficient athymic nude mice were inoculated subcutaneously with COLO205 (human colorectal adenocarcinoma) tumor cells. Tumors were allowed to establish and mice were sorted into five treatment groups of 6 mice per group. When the mean tumor volume reached an average size of 117 mm3 per group, each group was treated with one of the following compounds, administered intravenously at the indicated dosages: Group 1 (Vehicle; phosphate buffered saline (PBS)); Group 2 LY75_DM1; 10 mg/kg), Group 3 (Isotype control-DM1; 10 mg/kg), Group 4 (LY75_DM4; 5 mg/kg), Group 5 (Isotype control-DM4; 5 mg/kg). A second dose was administered twelve days after the first. Body weights (BW) were monitored, the mice were examined frequently for health and adverse side effects, and tumors were measured twice weekly. Mice were euthanized when their tumors reached the tumor volume endpoint of 1000 mm3 or after 60 days, whichever came first. Efficacy was determined from tumor growth delay (TGD), the increase in median time-to-endpoint (TTE) and from log rank analysis of differences in Kaplan Meier survival curves in ADC-treated versus PBS-treated mice. The first five vehicle-treated control mice to reach endpoint were sampled for tumors that were processed by formalin fixation and paraffin embedded.

### Results

FIG. 4f shows LY75_DM1 and LY75_DM4 exhibited similar modest anti-tumor activity and survival extension in the COLO205 colorectal adenocarcinoma nude mouse xenograft model compared to controls. All treatments were well-tolerated and no clinical signs of toxicity were observed. These data suggest the potential for ADCs directed towards LY75, for example LY75_DM1 and LY75_DM4, to provide clinical benefit in the treatment of human colorectal cancer patients.

### Example 21: Toxicity of DM1-Conjugated and DM4-Conjugated Anti-LY75 Monoclonal Antibodies in Cynomolgus Monkeys

Six male monkeys were assigned to the study with 2 monkeys/group. Either vehicle (PBS), LY75_DM4 (cleavable) or LY75_DM1 (non-cleavable) was administered twice (on Day 1 and Day 29) by a 15-minute intravenous infusion at 0 mg/kg/dose (PBS, vehicle), 5 mg/kg/dose (LY75_DM4, cleavable) or 10 mg/kg/dose (LY75_DM1, non-cleavable). Blood samples were collected for toxicokinetic evaluations prior to dose initiation (Day 1), and 1, 2, 3, 7, 14, 21 and 28 days post each dose. Blood samples for clinical pathology analyses were collected prior to dose initiation (Day 1), and 1, 3, 7, 14, 21 and 28 days post each dose (28 days post the 1st dose was also served as the pre-dose time point for the 2nd dose). All study animals were euthanized and necropsied following the final blood collection on Day 57. The plasma separated from each blood draw was isolated, frozen and shipped to Oxford BioTherapeutics, Inc. to be analyzed for ADC concentration by ELISA.

Treatment-related clinical pathology findings included a mild regenerative anemia and transient decreases in the blood leukocyte profile most notably in neutrophils counts. Anemia was observed in both animals treated with 5 mg/kg LY75_DM4 and in one of the two animals treated with 10 mg/kg LY75_DM1. Severe neutropenia with a nadir at one-week post dose and a rapid recovery in counts was observed in all animals; the nadir in absolute neutrophil count was lower in LY75_DM4 treated animals. There were no test article-related effects on the APTT and PT coagulation parameters. Serum chemistry changes included transient increases in AST, CK, LDH (in 1 of 2 animals in each treatment group) and globulin following administration of 5 mg/kg LY75_DM4 and 10 mg/kg LY75_DM1. In addition, a transient increase in the liver specific enzyme ALT was observed only in the LY75_DM4 treated animals. The short duration of and/or the magnitude of the increases in serum chemistry parameters suggest they were not adverse. There were no test-article related urinalysis findings. Upon examination at necropsy following a 4-week recovery period there were no treatment related gross pathology findings or changes in absolute and relative organ weights. Histopathology findings only in the thyroid gland (an alteration in the colloid morphology in follicles) and kidney (dilated tubules in the outer cortex), were graded as minimal severity; not associated with changes in other study parameters; and, not adverse and of minimal toxicological significance. Conclusion: Repeated dose treatment with two doses of 5 mg/kg LY75_DM4 or 10 mg/kg LY75_DM1 was well tolerated in cynomolgus monkeys. All treatment-related toxicity findings were reversible following a 4-week recovery period.

### Example 22 Synergistic Combinations of LY75 DM4 and Platin

### Method

Cells were diluted in excess media and spun down. 3000-10000 cells/well were plated in a white-walled, clear-bottom, TC-treated 96 well plate (Falcon: 353377), in 100 ul media/well. The plate was Incubated for 24 hr at 37° C. to allow cells to adhere and become acclimatized prior to addition of drug. The media was removed from the cells and 100 ul/well of appropriate dilutions of LY75_A1 was added as shown in the figures. The plates were incubated for 72 hr at 37° C. After 72 hr, the media was removed and replaced with either cisplatin or oxaliplatin at various concentrations (see figures) in 100 ul media before incubation for a further 48 hr.

After the incubation period, the plate was removed from the incubator and left at room temperature for 30 minutes. Media was removed from the plate which was washed twice with 200 ul PBS. The plate was then treated with cell-titer glo (CTG) following the manufacturer's protocol (Promega, Catalog No. G9681). Briefly, 200 ul CTG solution/well was added before incubating at RT on a plate rotator at low speed for 2 min before incubation in the dark for a further 10 min.

The plates were removed and the viability under the various conditions calculated.

### Results

Table 1 shows that in CRC, pancreatic and gastric cell lines the combination of a platin and LY75_DM4 was synergistic.

| | | | | | LY75 |
|---|---|---|---|---|---|
| | | Response | Response | | Antigen |
| | Cell | to | to | | Expression/ |
| Indication | Line | Platinum | LY75_DM4 | Combination | Cell |
| CRC | HT29 | Y | Y | Synergistic | 15,990 |
| Pancreatic | HPAFII | Y | Y | Synergistic | 28,180 |
| Gastric | N87 | Y | Y | Synergistic | 3,552 |

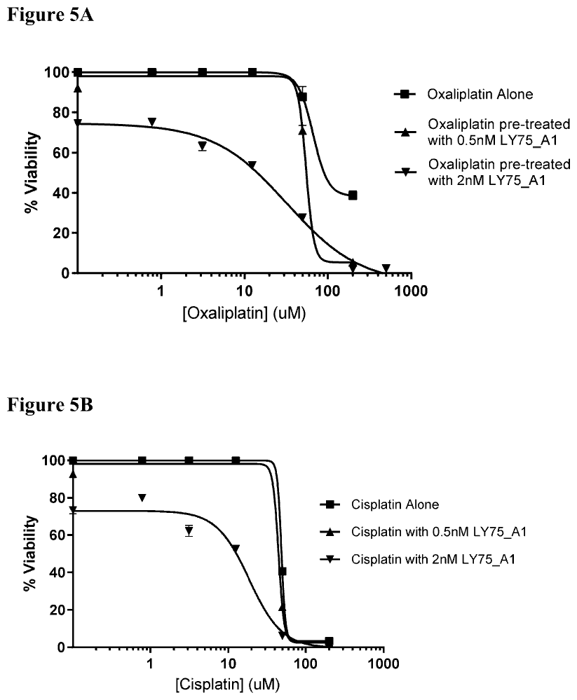

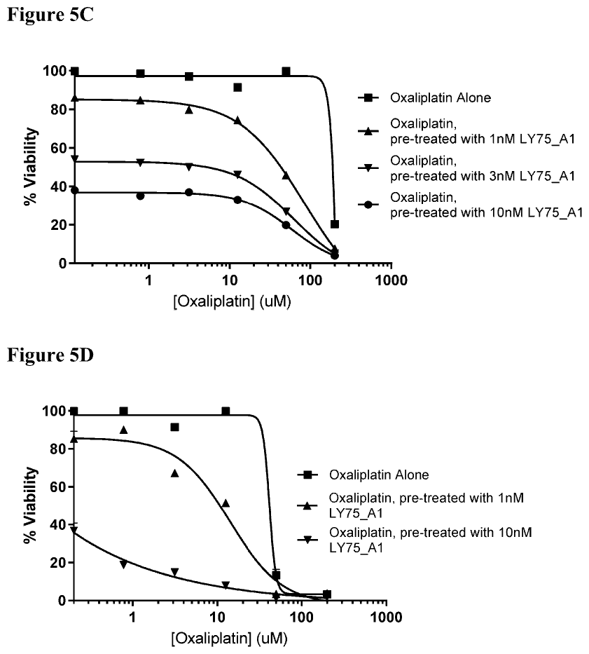

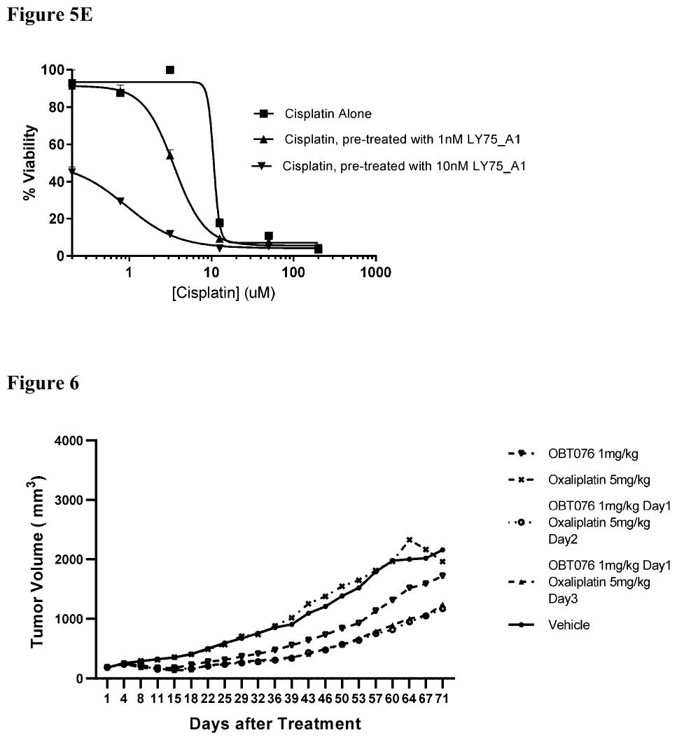

FIG. 5A shows the effect of treatment of HT-29 cells with either oxaliplatin alone or oxaliplatin on cells that have been pre-treated with 0.5 nM or 2 nM anti-LY75_A1 for 72 hrs. It can be seen that at ˜10 uM oxaliplatin the viability of cells pre-treated with 2 nM LY75_A1 is significantly lower than those which are not pre-treated which show no cell death.

Table 2 shows that in HT29 cells the IC50 (μM) for oxaliplatin halved in cells which have been pre-treated with LY75_A1 for 72 hours.

| Treatment | IC₅₀ (uM) |
|---|---|
| Oxaliplatin | 66.8 |
| Oxaliplatin pretreated with 0.5 nM LY75_A1 | 55.1 |
| Oxaliplatin pretreated with 2 nM LY75_A1 | 32.7 |

FIG. 5B shows the effect of treatment of HT-29 cells with either cisplatin alone or cisplatin on cells that have been pre-treated with 0.5 nM or 2 nM anti-LY75_A1 for 72 hrs. It can be seen that at ˜10 uM cisplatin the viability of cells pre-treated with 2 nM LY75_A1 is significantly lower than those which are not pre-treated which show no cell death.

Table 3 shows that in HT29 cells the IC50 (UM) for cisplatin more than halved in cells which have been pre-treated with LY75_A1 for 72 hours.

| Treatment | IC₅₀ (uM) |
|---|---|
| Cisplatin | 44.7 |
| Cisplatin pretreated with 0.5 nM LY75_A1 | 44.6 |
| Cisplatin pretreated with 2 nM LY75_A1 | 18.7 |

FIG. 5C shows the effect of treatment of HPAFII cells with either oxaliplatin alone or oxaliplatin on cells that have been pre-treated with 1 nM, 3 nM or 10 nM anti-LY75_A1 for 72 hrs. It can be seen that at ˜50 uM oxaliplatin the viability of cells pre-treated with all concentrations of LY75_A1 is significantly lower than those which are not pre-treated which show no cell death. Table 4 shows that in HPAFII cells the IC50 (UM) for oxaliplatin was reduced approximately 4 fold in cells which have been pre-treated with LY75_A1 for 72 hours.

| Treatment | IC₅₀ (uM) |
|---|---|
| Oxaliplatin | ~256.3 |
| Oxaliplatin pretreated with 1 nM LY75_A1 | 85.35 |
| Oxaliplatin pretreated with 3 nM LY75_A1 | 62.12 |
| Oxaliplatin pretreated with 10 nM LY75_A1 | 58.84 |

FIG. 5D shows the effect of treatment of N87 cells with either oxaliplatin alone or oxaliplatin on cells that have been pre-treated with 1 nM or 10 nM anti-LY75_A1 for 72 hrs. It can be seen that at ˜10 uM oxaliplatin the viability of cells pre-treated with 1 or 10 nM LY75_A1 is significantly lower than those which are not pre-treated which show no cell death.

Table 5 shows that in N87 cells the IC50 (UM) for oxaliplatin reduced 44 fold in cells which have been pre-treated with LY75_A1 for 72 hours.

| Treatment | IC₅₀ (uM) |
|---|---|
| Oxaliplatin | 44.5 |
| Oxaliplatin pretreated with 1 nM LY75_A1 | 14.1 |
| Oxaliplatin pretreated with 10 nM LY75_A1 | 1.0 |

FIG. 5E shows the effect of treatment of N87 cells with either cisplatin alone or cisplatin on cells that have been pre-treated with 1 nM or 10 nM anti-LY75_A1 for 72 hrs. It can be seen that at ˜3 uM cisplatin the viability of cells pre-treated with 1 or 10 nM LY75_A1 is significantly lower than those which are not pre-treated which show no cell death. Table 6 shows that in N87 cells the IC50 (UM) for cisplatin reduced 10 fold in cells which have been pre-treated with LY75_A1 for 72 hours.

| Treatment | IC₅₀ (uM) |
|---|---|
| Cisplatin | 10.0 |
| Cisplatin pretreated with 1 nM LY75_A1 | 3.5 |
| Cisplatin pretreated with 10 nM LY75_A1 | 0.9 |

As can be seen, in all cell lines pre-treatment with LY75_A1 significantly increases the sensitivity to platins. This can be seen to be synergistic because the combined cytotoxicity due to the single agents is less compared to when both drugs are used in combination.

### Example 23 In Vivo Efficacy Study of the Effect of OBT076 and Oxaliplatin in the Treatment of Subcutaneous NCI-N87 Gastric Cancer Xenograft Model

### Materials and Methods

### Mice

Female athymic nude mice (Balb-C,) ordered from GemPharmatech Co., Ltd (Nanjing, China) were 6-8 weeks old and had a BW range of 17.5-24.8 g on D1 of the study. The animals were fed ad libitum water (reverse osmosis, 1 ppm CI) and NIH 31 Modified and Irradiated Lab Diet® consisting standard rodent chow. The mice were housed on Crushed corncob bedding, autoclaved; changed weekly on a 12-hour light cycle at 20-22° C. (68-72° F.) and 40-60% humidity.

### Tumor Cell Culture

The NCI-N87 tumor cells were maintained in vitro with RMPI 1640 medium supplemented with 10% fetal bovine serum at 37° C. in an atmosphere of 5% CO₂ in air. The cells in exponential growth phase were harvested and quantitated by cell counter before tumor inoculation.

### Tumor Implantation and Growth

Each mouse was inoculated subcutaneously in the right upper flank region with NCI-N87 tumor cells (1×10⁷) in PBS mixed with matrigel (1:1) for tumor development. After tumor inoculation, the animals were checked daily for morbidity and mortality. During routine monitoring, the animals were checked for any effects of tumor growth and treatments on behavior such as mobility, food and water consumption, body weight gain/loss (Body weights would be measured two times per week after randomization), eye/hair matting and any other abnormalities. Mortality and observed clinical signs were recorded for individual animals in detail.

Tumor volumes were measured two times per week after randomization in two dimensions using a caliper, and the volume was expressed in mm3 using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

The body weights and tumor volumes were measured by using Study Director™ software (version 3.1.399.19).

The randomization started when the mean tumor size reached approximately 183 mm3. Totally 35 mice were enrolled in the study and randomly allocated to 5 study groups as shown in Table 7, with 7 mice per group. Randomization was performed based on “Matched distribution” method (Study Director™ software, version 3.1.399.19). The date of randomization was denoted as day 1.

### Test Articles

Dosing solutions were prepared once per study and were stored at 4° C., protected from light. Dosing volume was 10 ml/kg (0.200 mL/20 g mouse) and volumes were adjusted accordingly for actual body weight.

### Treatment

The treatment plan is shown in Table 7. Test articles and control were each administered via infusion vein injections (i.v.) on D1 for OBT076 and D1, D2 and D3 for Oxaliplatin. The dosing volume=10 μL/g was scaled to the most recent weights of individual animals.

TABLE 7
Treatment Plan for NCI-N87 CDX Model Study

| | | Dose | Dosing | Dosing | Dosing |
|---|---|---|---|---|---|
| | | Level | Solution | Volume | Frequency & |
| Group | Treatment | (mg/kg) | (mg/mL) | (mL/kg) | Duration |
| 1 | OBT076(day1) | 1.0 | 0.1 | 10 | single |
| 2 | Oxaliplatin(day1) | 5 | 0.5 | 10 | single |
| 3 | OBT076(day1) | 1.0 | 0.1 | 10 | single |
| | Oxaliplatin(day2) | 5 | 0.5 | 10 | single |
| 4 | OBT076 (day1) | 1.0 | 0.1 | 10 | single |
| | Oxaliplatin(day3) | 5 | 0.5 | 10 | single |
| 5 | — | — | — | — | — |
| | Vehicle (saline) | — | — | 10 | single |

### Tumor Growth Delay

Each animal was euthanized for tumor progression (TP) when its tumor reached the 3000 mm volume endpoint. The time to endpoint (TTE) for each mouse was calculated with the following equation: TTE=log (endpoint volume)−b/m. Where, b is the intercept and m is the slope of the line obtained by linear regression of a log-transformed tumor growth data set. The data set is comprised of the first observation that exceeded the study endpoint volume and the three consecutive observations that immediately preceded the attainment of the endpoint volume. Any animal that did not reach endpoint was euthanized at the end of the study and assigned a TTE value equal to the last day of the study. Any animal determined to have died from treatment-related (TR) causes was to be assigned a TTE value equal to the day of death. Any animal that died from non-treatment-related (NTR) causes was to be excluded from the analysis.

Treatment outcome was evaluated from tumor growth delay (TGD), which was defined as the increase in the median TTE for a treatment group compared to the control group: TGD=T−C expressed in days or as a percentage of the median TTE for the control group % TGD=T−C/C×100 where, T=median TTE for a treatment group, C=median TTE for the control group.

### Median Tumor Volume and Criteria for Regression Response

Treatment efficacy may also be determined from the tumor volumes of animals remaining in the study on the last day and from the number of regression responses. The MTV (n) was defined as the median tumor volume in the number of animals remaining, n, whose tumors had not attained the volume endpoint.

Treatment may cause partial regression (PR) or complete regression (CR) of the tumor in an animal. In a PR response, the tumor volume is 50% or less of its D1 volume for three consecutive measurements during the course of the study, and equal to or greater than 13.5 mm3 for one or more of these three measurements. In a CR response, the tumor volume is less than 13.5 mm3 for three consecutive measurements during the course of the study.

### Toxicity

Animals were weighed daily on Days 1-5, and then twice weekly until the end of the study. Group mean BW nadirs were determined before more than 50% of the animals in a group exited the study. The mice were observed frequently for health and overt signs of any adverse TR side effects, and clinical observations were recorded, if observed. Acceptable toxicity was defined as a group mean BW loss of less than 20% during the study and not more than 10% TR death. Any dosing regimen resulting in greater toxicity is considered above the maximum tolerated dose (MTD). A death was to be classified as TR if it was attributable to treatment side effects as evidenced by clinical signs and/or necropsy, or due to unknown causes during the dosing period or within 14 days after the last dose. Any animal with weight loss exceeding 30% for one measurement, or exceeding 25% for three measurements, was to be euthanized for health as a TR death. A death was to be classified as NTR if there was no evidence that the death was related to treatment side effects and occurred more than 14 days post dosing. NTR deaths were to be further categorized as NTRa (due to accident or human error), NTRm (due to necropsy-confirmed tumor dissemination by invasion or metastasis), or NTRu (due to unknown causes).

TABLE 8
Tumor growth inhibition (TGI) results Calculated based on
the tumor size on day 60 (last day that contains all mice data).

| | Treatment | Tumor Size (mm³) ^{a} | TGI | |
|---|---|---|---|---|
| Group | Description | on day 60 | on day 60 | P value |
| 1 | OBT076(day1), | 1311.25 +/− 260.64 | 33.76% | 0.398 |
| | 1 mg/kg, 10 ul/g | | | |
| 2 | Oxaliplatin(day1), | 1969.92 +/− 258.93 | 0.49% | 1 |
| | 5 mg/kg, 10 ul/g | | | |
| 3 | OBT076(day1), | 817.08 +/− 178.57 | 58.72% | 0.00361 |
| | 1 mg/kg, 10 ul/g, | | | |
| | Oxaliplatin(day2), | | | |
| | 5 mg/kg, 10 ul/g | | | |
| 4 | OBT076(day1), | 894.66 +/− 148.60 | 54.81% | 0.00913 |
| | 1 mg/kg, 10 ul/g, | | | |
| | Oxaliplatin(day3), | | | |
| | 5 mg/kg, 10 ul/g | | | |
| 5 | Vehicle (saline), | 1979.58 +/− 300.86 | — | — |
| | 10 ul/g | | | |

### Statistical Analysis

To compare tumor volumes of different groups at a pre-specified day, we first used Bartlett's test to check the assumption of homogeneity of variance across all groups. When the p-value of Bartlett's test is >0.05, we ran one-way ANOVA to test overall equality of means across all groups. If the p-value of the one-way ANOVA is <0.05, we further performed post hoc testing by running Tukey's HSD (honest significant difference) tests for all pairwise comparisons, and Dunnett's tests for comparing each treatment group with the vehicle group. When the p-value of Bartlett's test was <0.05, we ran Kruskal-Wallis test to test overall equality of medians among all groups. If the p-value the Kruskal-Wallis test was <0.05, we further performed post hoc testing by running Conover's non-parametric test for all pairwise comparisons or for comparing each treatment group with the vehicle group, both with single-step p-value adjustment. All statistical analyses were done in R-a language and environment for statistical computing and graphics (version 3.3.1). All tests were two-sided unless otherwise specified, and p-values of <0.05 were regarded as statistically significant.

As can be seen from Table 8 and FIG. 6 the mice administered the combination of OBT076 (anti LY75) and Oxaliplatin showed significantly improved tumor growth delay compared to the mice administered either OBT076 or oxaliplatin alone.

Furthermore, as seen from Table 9 below the mice administered the combination of OBT076 and Oxaliplatin had all survived to Day 81, when the study was ended, whereas in the groups of mice administered either OBT076 or oxaliplatin alone ˜29% and ˜71% respectively had died.

TABLE 9

| Group | Treatment Description | % Survival |
|---|---|---|
| 1 | OBT076(day1), 1 mg/kg, 10 ul/g | 71.43% |
| 2 | Oxaliplatin(day1), 5 mg/kg, 10 ul/g | 28.57% |
| 3 | OBT076(day1), 1 mg/kg, 10 ul/g, | 100% |
| | Oxaliplatin(day2), 5 mg/kg, 10 ul/g | |
| 4 | OBT076(day1), 1 mg/kg, 10 ul/g, | 100% |
| | Oxaliplatin(day3), 5 mg/kg, 10 ul/g | |
| 5 | Vehicle (saline), 10 ul/g | 42.86% |

### Sequences

| SEQ ID | | |
|---|---|---|
| No | Description | Sequence |
| 1 | A1_VH aa | EVQLVESGGGLVKPGGSLRLSCAASGFTYSNAWMSWVRQAPGK |
| | | GLEWVGRIKSKTDGGTTDYAAPVQGRFTISRDDSKNTLYLQMN |
| | | SLKTEDTAVYYCTIFGVVSFDYWGQGTLVTVSS |
| 2 | A1_VL aa | DVQMTQSPSSLSASVGDRVTITCRASQSISDYLSWYQQRPGKA |
| | | PNLLIYAASNLKTGVPSRFSGSGSGTDFTLTISTLQPEDFATY |
| | | YCQQSYRSPWTFGQGTKVEIKR |
| 3 | A1_VH nt | gaggtgcagctggtggagtctgggggaggcttggtaaagccgg |
| | | gggggtcccttagactctcctgtgcagcctctggcttcactta |
| | | cagtaacgcctggatgagctgggtccgccaggctccagggaag |
| | | gggctggagtgggttggccgtattaaaagcaaaactgatggtg |
| | | ggacaacagactacgctgcacccgtgcaaggcagattcaccat |
| | | ctcaagagatgattcaaaaaacacgctgtatctgcaaatgaac |
| | | agcctgaaaaccgaggacacagccgtgtattactgtacgattt |
| | | ttggagtggttagctttgactactggggccagggaaccctggt |
| | | caccgtctcctca |
| 4 | A1_VL nt | gacgtccagatgacccagtctccatcctccctgtctgcatctg |
| | | ttggagacagagtcaccatcacttgccgggcaagtcagagcat |
| | | tagcgactatttaagttggtatcagcagagaccagggaaagcc |
| | | cctaacctcctgatctatgctgcatccaatttaaagactgggg |
| | | tcccatcaaggttcagtggcagtggatctgggacagatttcac |
| | | tctcaccatcagcactctgcaacctgaagattttgcaacgtac |
| | | tactgtcaacagagttacaggtccccgtggacgttcggccaag |
| | | ggaccaaggtggaaatcaaacga |
| 5 | A1_VH_CDR1 | NAWMS |
| | aa | |
| 6 | A1_VH_CDR2 | RIKSKTDGGTTDYAAPVQG |
| | aa | |
| 7 | A1_VH_CDR3 | FGVVSFDY |
| | aa | |
| 8 | A1_VL_CDR1 | RASQSISDYLS |
| | aa | |
| 9 | A1_VL_CDR2 | AASNLKT |
| | aa | |
| 10 | A1_VL_CDR3 | QQSYRSPWT |
| | aa | |
| 11 | VH3\|3-15/D4\| | EVQLVESGGGLVKPGGSLRLSCAASGFTFSNAWMSWVRQAPGK |
| | 411 | GLEWVGRIKSKTDGGTTDYAAPVKGRFTISRDDSKNTLYLQMN |
| | | SLKTEDTAVYYCTTTTVT |
| 12 | JH4 | YFDYWGQGTLVTVSS |
| 13 | O12 | DIQMTQSPSSLSASVGDRVTITCRASQSISSYLNWYQQKPGKA |
| | | PKLLIYAASSLQSGVPSRFSGSGSGTDFTLTISSLQPEDFATY |
| | | YCQQSYS |
| 14 | JK1 | WTFGQGTKVEIKR |
| 15 | LY75 | MRTGWATPRRPAGLLMLLFWFFDLAEPSGRAANDPFTIVHGNT |
| | (DEC-205) | GKCIKPVYGWIVADDCDETEDKLWKWVSQHRLFHLHSQKCLGL |
| | | DITKSVNELRMFSCDSSAMLWWKCEHHSLYGAARYRLALKDGH |
| | | GTAISNASDVWKKGGSEESLCDQPYHEIYTRDGNSYGRPCEFP |
| | | FLIDGTWHHDCILDEDHSGPWCATTLNYEYDRKWGICLKPENG |
| | | CEDNWEKNEQFGSCYQFNTQTALSWKEAYVSCQNQGADLLSIN |
| | | SAAELTYLKEKEGIAKIFWIGLNQLYSARGWEWSDHKPLNFLN |
| | | WDPDRPSAPTIGGSSCARMDAESGLWQSFSCEAQLPYVCRKPL |
| | | NNTVELTDVWTYSDTRCDAGWLPNNGFCYLLVNESNSWDKAHA |
| | | KCKAFSSDLISIHSLADVEVVVTKLHNEDIKEEVWIGLKNINI |
| | | PTLFQWSDGTEVTLTYWDENEPNVPYNKTPNCVSYLGELGQWK |
| | | VQSCEEKLKYVCKRKGEKLNDASSDKMCPPDEGWKRHGETCYK |
| | | IYEDEVPFGTNCNLTITSRFEQEYLNDLMKKYDKSLRKYFWTG |
| | | LRDVDSCGEYNWATVGGRRRAVTFSNWNFLEPASPGGCVAMST |
| | | GKSVGKWEVKDCRSFKALSICKKMSGPLGPEEASPKPDDPCPE |
| | | GWQSFPASLSCYKVFHAERIVRKRNWEEAERFCQALGAHLSSF |
| | | SHVDEIKEFLHFLTDQFSGQHWLWIGLNKRSPDLQGSWQWSDR |
| | | TPVSTIIMPNEFQQDYDIRDCAAVKVFHRPWRRGWHFYDDREF |
| | | IYLRPFACDTKLEWVCQIPKGRTPKTPDWYNPDRAGIHGPPLI |
| | | IEGSEYWFVADLHLNYEEAVLYCASNHSFLATITSFVGLKAIK |
| | | NKIANISGDGQKWWIRISEWPIDDHFTYSRYPWHRFPVTFGEE |
| | | CLYMSAKTWLIDLGKPTDCSTKLPFICEKYNVSSLEKYSPDSA |
| | | AKVQCSEQWIPFQNKCFLKIKPVSLTFSQASDTCHSYGGTLPS |
| | | VLSQIEQDFITSLLPDMEATLWIGLRWTAYEKINKWTDNRELT |
| | | YSNFHPLLVSGRLRIPENFFEEESRYHCALILNLQKSPFTGTW |
| | | NFTSCSERHFVSLCQKYSEVKSRQTLQNASETVKYLNNLYKII |
| | | PKTLTWHSAKRECLKSNMQLVSITDPYQQAFLSVQALLHNSSL |
| | | WIGLFSQDDELNFGWSDGKRLHFSRWAETNGQLEDCVVLDTDG |
| | | FWKTVDCNDNQPGAICYYSGNETEKEVKPVDSVKCPSPVLNTP |
| | | WIPFQNCCYNFIITKNRHMATTQDEVHTKCQKLNPKSHILSIR |
| | | DEKENNFVLEQLLYFNYMASWVMLGITYRNKSLMWFDKTPLSY |
| | | THWRAGRPTIKNEKFLAGLSTDGFWDIQTFKVIEEAVYFHQHS |
| | | ILACKIEMVDYKEEYNTTLPQFMPYEDGIYSVIQKKVTWYEAL |
| | | NMCSQSGGHLASVHNQNGQLFLEDIVKRDGFPLWVGLSSHDGS |
| | | ESSFEWSDGSTFDYIPWKGQTSPGNCVLLDPKGTWKHEKCNSV |
| | | KDGAICYKPTKSKKLSRLTYSSRCPAAKENGSRWIQYKGHCYK |
| | | SDQALHSFSEAKKLCSKHDHSATIVSIKDEDENKFVSRLMREN |
| | | NNITMRVWLGLSQHSVDQSWSWLDGSEVTFVKWENKSKSGVGR |
| | | CSMLIASNETWKKVECEHGFGRVVCKVPLGPDYTAIAIIVATL |
| | | SILVLMGGLIWFLFQRHRLHLAGFSSVRYAQGVNEDEIMLPSF |
| | | HD |
| 16 | A1_VH_FR1 | EVQLVESGGGLVKPGGSLRLSCAASGFTYS |
| 17 | A1_VH_FR2 | WVRQAPGKGLEWVG |
| 18 | A1_VH_FR3 | RFTISRDDSKNTLYLQMNSLKTEDTAVYYCTI |
| 19 | A1_VH_FR4 | WGQGTLVTVSS |
| 20 | A1_VL_FR1 | DVQMTQSPSSLSASVGDRVTITC |
| 21 | A1_VL_FR2 | WYQQRPGKAPNLLIY |
| 22 | A1_VL_FR3 | GVPSRFSGSGSGTDFTLTISTLQPEDFATYYC |
| 23 | A1_VL_FR4 | FGQGTKVEIKR |
| 24 | A1_H | MEWSWVFLFFLSVTTGVHSEVQLVESGGGLVKPGGSLRLSCAA |
| | (amino acid) | SGFTYSNAWMSWRQAPGKGLEWVGRIKSKTDGGTTDYAAPVQG |
| | | RFTISRDDSKNTLYLQMNSLKTEDTAVYYCTIFGVVSFDYWGQ |
| | | GTLVTVSSASTKGPSVFPLAPSSKSTSGGTAALGCLVKDYFPE |
| | | PVTVSWNSGALTSGVHTFPAVLQSSGLYSLSSVVTVPSSSLGT |
| | | QTYICNVNHKPSNTKVDKKVEPKSCDKTHTCPPCPAPELLGGP |
| | | SVFLFPPKPKDTLMISRTPEVTCVVVDVSHEDPEVKFNWYVDG |
| | | VEVHNAKTKPREEQYNSTYRVVSVLTVLHQDWLNGKEYKCKVS |
| | | NKALPAPIEKTISKAKGQPREPQVYTLPPSREEMTKNQVSLTC |
| | | LVKGFYPSDIAVEWESNGQPENNYKTTPPVLDSDGSFFLYSKL |
| | | TVDKSRWQQGNVFSCSVMHEALHNHYTQKSLSLSPGK |
| 25 | A1_L | MSVPTQVLGLLLLWLTDARCDVQMTQSPSSLSASVGDRVTITC |
| | (amino acid) | RASQSISDYLSWYQQRPGKAPNLLIYAASNLKTGVPSRFSGSG |
| | | SGTDFTLTISTLQPEDFATYYCQQSYRSPWTFGQGTKVEIKRT |
| | | VAAPSVFIFPPSDEQLKSGTASVVCLLNNFYPREAKVQWKVDN |
| | | ALQSGNSQESVTEQDSKDSTYSLSSTLTLSKADYEKHKVYACE |
| | | VTHQGLSSPVTKSFNRGEC |
| 26 | A1_H (nt) | atggaatgga gctgggtgtt cctgttcttt ctgtccgtga |
| | | ccacaggcgt gcattctgaa gttcagctgg tcgaaagcgg |
| | | aggaggtctg gtgaaacccg gtggctccct gaggctgagc |
| | | tgcgccgcct ccggctttac ttacagtaat gcctggatgt |
| | | cctgggtcag acaggcccca ggtaagggtc tggagtgggt |
| | | gggtaggatt aagtctaaaa ctgatggcgg gacaacagac |
| | | tatgccgccc cagtgcaagg acggttcacc atttctaggg |
| | | acgactctaa gaatacactg tatctgcaga tgaacagcct |
| | | caaaacagaa gacactgccg tttactactg taccatcttt |
| | | ggcgttgtct cctttgatta ttggggacag ggtacactcg |
| | | tgaccgtttc ttccgcaagt acaaaggggc catcagtgtt |
| | | tccactggcc ccatcctcta agagcactag tggcggcaca |
| | | gccgccctgg gatgtctggt gaaggactat ttcccagagc |
| | | ctgtgaccgt cagctggaac agtggtgctc tcacctcagg |
| | | tgtgcacaca ttccccgctg tgctccaatc cagtggcctc |
| | | tacagtctga gcagcgttgt gactgttccc agtagctcac |
| | | tgggcaccca aacctacata tgcaatgtga accataaacc |
| | | tagcaatacc aaagtggaca agaaagtgga acctaagtcc |
| | | tgtgacaaga ctcatacctg tcctccttgt cctgccccag |
| | | agctgctcgg aggcccttcc gtctttctct tcccaccaaa |
| | | gccaaaggat accctgatga tcagccggac acctgaggtt |
| | | acctgcgttg tggtcgacgt ttcacacgag gatcctgaag |
| | | tcaaattcaa ctggtacgtt gatggagtcg aggtccacaa |
| | | cgccaaaacc aagcctcgcg aagaacaata caatagcaca |
| | | tatagggtgg tgtctgtgct cactgtcctg caccaggact |
| | | ggctgaacgg caaggagtac aaatgcaagg ttagtaacaa |
| | | ggccctgccc gcacccattg agaagactat cagtaaagct |
| | | aagggccagc ctcgcgagcc tcaggtttac accctgcctc |
| | | cctctagaga ggaaatgaca aagaaccagg tgtctctcac |
| | | ctgcctggtt aaaggattct atccatccga cattgctgtg |
| | | gaatgggaat ccaacggaca gcccgaaaac aactataaga |
| | | caacaccacc tgttctggat tccgatggtt ccttctttct |
| | | gtattccaaa ctcacagtgg acaagagtcg ctggcagcaa |
| | | ggtaacgtgt tttcttgctc cgtgatgcac gaagcactcc |
| | | acaatcacta cactcagaag agtctcagcc tctctccagg |
| | | caaa |
| 27 | A1_L1 (nt) | atgtctgtgc ctacccaggt gctgggactg ctgctgctgt |
| | | ggctgacaga cgcccgctgt gatgttcaga tgacacagtc |
| | | tccaagtagt ctcagcgcaa gcgttggcga cagagtgact |
| | | atcacatgca gagcctctca gtctatctct gactatctgt |
| | | cttggtacca gcagaggcca ggcaaagctc caaacctcct |
| | | gatctatgct gccagtaatc tgaagacagg cgtgcctagt |
| | | agattctccg ggtccggtag tgggactgat ttcaccctga |
| | | caatctccac actgcaacct gaggattttg ctacctacta |
| | | ttgtcagcaa tcttatcgca gcccttggac cttcggacag |
| | | gggactaagg ttgagattaa acgcaccgtg gcagcaccca |
| | | gcgtctttat ctttcctccc tccgacgage agctcaagtc |
| | | cggaacagca tcagtcgttt gcctcctgaa taacttttat |
| | | ccaagggagg ccaaggtcca gtggaaagtc gacaatgccc |
| | | tccaatctgg taactcccag gagtctgtga ctgaacaaga |
| | | ttctaaggac agtacctatt cactcagctc caccctgacc |
| | | ctcagcaaag cagactacga aaagcataaa gtttacgctt |
| | | gcgaagtgac ccaccaaggc ctgtcttctc ctgtcacaaa |
| | | gagttttaat agaggggagt gt |
| 28 | Linker | Gly-Phe-Leu-Gly |

1. A pharmaceutical combination comprising:
a) an anti-LY75 antibody, or an antigen-binding portion thereof, and
b) a platin drug or a pharmaceutically-acceptable salt thereof.

2. The pharmaceutical combination according to claim 1, wherein the pharmaceutical combination is in the form of a combined preparation for simultaneous, separate or sequential use.

3. The pharmaceutical combination according to claim 1, wherein said antibody or antigen binding portion thereof comprises:
a heavy chain variable region comprising:
i) a first vhCDR comprising SEQ ID NO: 5;
ii) a second vhCDR comprising SEQ ID NO: 6; and
iii) a third vhCDR comprising SEQ ID NO: 7; and
a light chain variable region comprising:
i) a first vICDR comprising SEQ ID NO: 8;
ii) a second vICDR comprising SEQ ID NO: 9; and
iii) a third vICDR comprising SEQ ID NO: 10;
optionally wherein any one or more of the above SEQ ID NOs independently comprise one, two, three, four or five amino acid substitutions, additions or deletions.

4. The pharmaceutical combination according to claim 3, wherein any one or more of SEQ ID NOs: 5-10 independently comprise one, two, three, four or five conservative amino acid substitutions.

5. The pharmaceutical combination according to claim 4, wherein any one or more of SEQ ID NOs: 5-10 independently comprise one or two conservative amino acid substitutions.

6. The pharmaceutical combination according to claim 1, wherein the anti-LY75 antibody or an antigen-binding portion thereof comprises:
(i) a heavy chain variable region having at least 80%, 85%, 90%, 95%, 99% or 100% amino acid sequence identity to SEQ ID NO: 1; and
(ii) a light chain variable region having at least 80%, 85%, 90%, 95%, 99% or 100% amino acid sequence identity to SEQ ID NO: 2.

7. The pharmaceutical combination according to claim 1, wherein the anti-LY75 antibody comprises:
(i) a heavy chain having at least 80%, 85%, 90%, 95%, 99% or 100% amino acid sequence identity to SEQ ID NO: 24; and
(ii) a light chain having at least 80%, 85%, 90%, 95%, 99% or 100% amino acid sequence identity to SEQ ID NO: 25.

8. The pharmaceutical combination according to claim 1, wherein the anti-LY75 antibody is a human IgG 1 monoclonal antibody.

9. The pharmaceutical combination according to claim 1, wherein the platin drug is selected from the list comprising cisplatin, carboplatin, oxaliplatin, nedaplatin, lobaplatin and heptaplatin.

10. The pharmaceutical combination according to claim 1, wherein the anti-LY75 antibody or an antigen-binding portion thereof further comprises a covalently-attached moiety.

11. The pharmaceutical combination according to claim 10, wherein said moiety is a cytotoxic moiety, preferably a drug.

12. The pharmaceutical combination according to claim 11, wherein said drug is a maytansinoid, a dolastatin, a hemiasterlin, an auristatin, a trichothecene, a calicheamicin, a duocarmycin, a bacterial immunotoxin, a pyranoindoizinoquinoline, a camptothecin, an anthracycline, an antheamycin, a thienoindole, an indolino-benzodiazepine, an amatoxin, CC1065 or taxol and derivatives thereof.

13. The pharmaceutical combination according to claim 12, wherein said drug is a maytansinoid selected from the group consisting of DM4 or DM1, preferably DM4.

14. The pharmaceutical combination according to claim 1, additionally comprising one or more pharmaceutically-acceptable diluents, excipients or carriers.

15. A method of treating cancer in a patient comprising administering to the patient the pharmaceutical combination of claim 1.

16. The method of claim 15, wherein said cancer is a LY75 positive cancer.

17. The method of claim 15, wherein the cancer is selected from the list consisting of comprising pancreatic cancer, ovarian cancer, breast cancer, endometrial cancer, gastroesophageal junction cancer, colorectal cancer, esophageal cancer, skin cancer, thyroid cancer, lung cancer (NSCLC and/or SCLC), kidney cancer, liver cancer, head and neck cancer, bladder cancer, gastric cancer, leukaemia, preferably acute myeloid leukaemia or chronic lymphocytic leukaemia, myeloma, preferably multiple myeloma and lymphoma, preferably diffuse large B-cell lymphoma (DLBCL), B-Cell Lymphoma, Follicular Lymphoma, Mantle Cell Lymphoma, Lymphoma of Mucosa-Associated Lymphoid Tissue (MALT), T-Cell/Histiocyte-Rich B-Cell Lymphoma, Burkitt's Lymphoma, Lymphoplasmacytic Lymphoma, Small Lymphocytic Lymphoma, Marginal Zone Lymphoma, T Cell Lymphoma, Peripheral T-Cell Lymphoma, Anaplastic Large Cell Lymphoma and Angiolmmunoblastic T-Cell Lymphoma.

18. The method of claim 17 wherein the cancer is selected from the list comprising gastric cancer, endometrial cancer, gastroesophageal junction cancer, colorectal cancer, bladder cancer, breast cancer, ovarian cancer, esophageal cancer, renal cancer, pancreatic cancer and lung cancer.

19. The method of claim 18, wherein the anti-LY75 antibody or antigen-binding portion thereof is internalized by a cell expressing LY75.

20. The method of claim 15, wherein the patient is a human.

21. The method of claim 15, wherein the platin drug is administered, 1 day, 2 days, 3, days, 4 days, 5 days, 6 days, 7 days, 8 days, 9 days, 10 days, 2 weeks, or 3 weeks after administration of the antibody or antigen binding portion thereof which binds to LY75, preferably 2 or 3 days.

22.-50. (canceled)
